(19) United States
(12) Patent Application Publication
(10) Pub. No.: US 2011/0038945 A1
(43) Pub. Date: Feb. 17, 2011

(54) Orally ingestable medicament and method for treating a heartburn inducing event or an acid reflux episode in a living human subject

(76) Inventor: Gavin M. Gear, Monterey, CA (US)

Correspondence Address: DAVID PRASHKER, P.C., P.O. Box 5387, Magnolia, MA, 01930, US

(21) Appl. No.: 12/583,041

(22) Filed: Aug. 13, 2009

(51) Int. Cl. A61K 35/56; A61P 1/04

(52) U.S. Cl. 424/537

(57) ABSTRACT

The present invention is a medicament, which is orally ingested by a living human subject for the prophylactic or therapeutic treatment of a heartburn inducing event or an acid reflux episode (GERD). In the preferred embodiments, the complete medicament is a fluid blending of at least one concentrated vinegar made by the fermentation of a fruit or fruit sugar, or a vegetable carbohydrate or sugar, or a grain carbohydrate or sugar; an undiluted bioactive honey having unique, non-peroxide antibacterial activity; a natural flavoring agent or combination of different natural flavors to neutralize the taste of the concentrated vinegar; and a natural sweetener to give the fluid blending a palatable taste. In these formulations, the concentrated vinegar serves to treats the symptoms of the heartburn and/GERD; and the undiluted bioactive honey employs its non-peroxide antibacterial activity to treat inflammation of the esophagus and infections of the stomach.

### FIELD OF THE INVENTION

The present invention is concerned generally with preventative and remedial treatments for heartburn and/or acid reflux disease in humans; and is specifically directed to medicinal compositions and methods for the alleviation of symptoms resulting from both acute and chronic heartburn as well as gastric and esophageal reflux disorder.

### BACKGROUND OF THE INVENTION

Gastro-esophageal reflux disorder (commonly referred to as “GERD” or acid reflux disease) is a human pathological condition in which the harsh liquid contents of the stomach become regurgitated or are refluxed upwards into the esophagus. The refluxed stomach liquids typically inflame and often can damage the cellular lining of the esophagus, although clearly visible signs of such inflammation occur only in a minority of patients.

The regurgitated stomach liquids usually contain both concentrated acid and pepsin, products that are produced by the stomach. Pepsin is an enzyme that begins the digestion of proteins in the stomach; and concentrated hydrochloric acid is a necessary component for human digestion. The refluxed liquids also may contain bile that has backed-up into the stomach from the duodenum. Anatomically, the duodenum is the first part of the small intestine that attaches to the stomach.

Among these regurgitated contents, the acid is believed to be the most injurious component. Pepsin and bile also may injure the esophagus, but their role in the production of esophageal inflammation and cellular damage is not as clear as the role of acid.

For greater medical details, the following scientific publications may be consulted. Kahrilas, P J. Diagnosis of symptomatic gastroesophageal reflux disease. Am J Gastroenterol 2003; 98: S15-S23; Pace, F, Santalucia, F, Bianchi, P G. Natural history of gastroesophageal reflux disease without esophagitis. Gut 1991; 32: 845-848; Trimble, K C, Douglas, S, Pryde, A, et al. Clinical characteristics and natural history of symptomatic but not excessive gastroesophageal reflux. Dig Dis Sci 1995; 40: 1098-1104; Tew, S, Jamieson, G G, Pilowski, I, et al. The illness behavior of patients with gastroesophageal reflux disease with and without endoscopic esophagitis. Dis Esophagus 1997; 10: 9-15; Mattox, H E, Richter, J E, Prolonged ambulatory esophageal pH monitoring in the evaluation of gastroesophageal reflux disease. Am J Med 1990; 89: 345-356; Weiner, G J, Morgan, J M, Copper, J B, et al. Ambulatory 24 hour esophageal pH monitoring: Reproducibility and variability of pH parameters. Dig Dis Sci 1988; 33: 1127-1133; Schlesinger, P K, Donahue, P E, Schmidt, B, et al. Limitations of 24 hour intraesophageal pH monitoring in the hospital setting. Gastroenterology 1985; 89: 797-804; Murphy, D W, Yuan, Y, Castell, D O. Does the intraesophageal pH probe accurately detect acid reflux? Simultaneous recording with two pH probes in humans. Dig Dis Sci 1989; 34: 649-656; Booth, M I, Stratford, 3, Dehn, T C B. Patient self-assessment of test-day symptoms in 24-h pH-metry for suspected gastroesophageal reflux disease. Scand J Gastroenterol 2001; 36: 795-799;

### A. Heartburn And GERD

Heartburn is a burning pain sensation behind the breastbone, and typically affects an estimated 20 percent of Americans at least once a week. While an occasional heartburn episode may be common, some people have heartburn frequently.

In addition, regular or constant heartburn is a common symptom of gastro-esophageal reflux disease (GERD). Other classic symptoms of GERD include those listed by Table A below.

TABLE A

Heartburn;
Chest pain, especially while lying down;
Sour taste in the mouth;
Coughing, wheezing, hoarseness & sore throat;
Regurgitation of food or liquid.

More detailed information about gastro-esophageal reflux disorder is available in the scientific and medical literature, of which some illustrative and representative examples include: DeVault, K R, Castell, D O. Guidelines for the diagnosis and treatment of gastroesophageal reflux disease. Arch Intern Med 1995; 155: 2165-2173; DeVault, K R, Castell, D O. Updated guidelines for the diagnosis and treatment of gastroesophageal reflux disease. Am 3 Gastroenterol 1999; 94: 1434-1442; Klauser, A G, Schindbeck, N E, Muller-Lissner, S A. Symptoms in gastro-esophageal reflux disease. Lancet 1990; 135: 205-208; Terea, L, Fein, M, Ritter, M P, et al. Can the combination of symptoms and endoscopy confirm the presence of gastroesophageal reflux disease? Am Surg 1997; 63: 933-936. Koehler, R E, Weymean, P J, Oakley, H F. Single- and double-contrast techniques in esophagitis. Am J Roentgenol 1980; 135: 15-19; Ott, D J, Chen, Y M, Felfand, D W, et al. Analysis of a multiphasic radiographic examination for detecting reflux esophagitis. Gastrointest Radiol 1986; 11: 1-6; Creteur, V, Thoeni, R F, Federle, M P, et al. The role of single- and double-contrast radiography in the diagnosis of reflux esophagitis. Radiology 1983; 147: 71-75; Ott, D J, Wu, W C, Gelfand, D W. Reflux esophagitis revisited: Prospective analysis of radiological accuracy. Gastrointest Radiol 1981; 6: 1-7; Sellan, R J, DeCaestecker, J S, Heading, R C. Barium radiology: A sensitive test for gastro-oesophageal reflux. Clin Radiol 1987; 38: 303-307; Johnston, B T, Troshinsky, M B, Castell, J A, et al. Comparison of barium radiology with esophageal pH monitoring in the diagnosis of gastroesophageal reflux disease. Am J Gastroenterol 1996; 91: 1181-1185; Sampliner, R E. Updated guidelines for the diagnosis, surveillance and therapy of Barrett's esophagus. Am J Gastroenterol 2002; 97: 1888-1895; Pace, F, Santalucia, F, Bianchi, P G. Natural history of gastroesophageal reflux disease without esophagitis. Gut 1991; 32: 845-848; Trimble, K C, Douglas, S, Pryde, A, et al. Clinical characteristics and natural history of symptomatic but not excessive gastroesophageal reflux. Dig Dis Sci 1995; 40: 1098-1104; Tew, S, Jamieson, G G, Pilowski, I, et al. The illness behavior of patients with gastroesophageal reflux disease with and without endoscopic esophagitis. Dis Esophagus 1997; 10: 9-15; Mattox, H E, Richter, J E, Prolonged ambulatory esophageal pH monitoring in the evaluation of gastroesophageal reflux disease. Am J Med 1990; 89: 345-356; Weiner, G J, Morgan, J M, Copper, J B, et al. Ambulatory 24 hour esophageal pH monitoring: Reproducibility and variability of pH parameters. Dig Dis Sci 1988; 33: 1127-1133; Schlesinger, P K, Donahue, P E, Schmidt, B, et al. Limitations of 24 hour intraesophageal pH monitoring in the hospital setting. Gastroenterology 1985; 89: 797-804; Murphy, D W, Yuan, Y, Castell, D O. Does the intraesophageal pH probe accurately detect acid reflux? Simultaneous recording with two pH probes in humans. Dig Dis Sci 1989; 34: 649-656; Booth, M I, Stratford, J, Dehn, T C B. Patient self-assessment of test-day symptoms in 24-h pH-metry for suspected gastroesophageal reflux disease. Scand J Gastroenterol 2001; 36: 795-799; Sifrim, D, Holloway, R, Silny, 3, et al. Acid, nonacid, and gas reflux in patients with gastroesophageal reflux disease during ambulatory 24-hour pH-impedance recordings. Gastroenterology 2001; 120: 1588-1598.

### B. GERD is a Chronic Condition

Gastro-Esophageal reflux disorder (“GERD”) is recognized as being a chronic pathological condition. Once regurgitation of the stomach liquids begins, the act of acid reflux usually is a life-long problem. Moreover, if there is subsequent injury to the cellular lining of the esophagus (the inflammation termed “esophagitis”), this type of injury is similarly a chronic condition.

Unfortunately also, even after the esophagus has become healed by effective medical treatment, when the treatment regimen is ended, the underlying causes of acid reflux remain. Thus, new and more serious injury to the esophagus will occur for most patients within a few months time after the initial treatment has stopped. For this reason, once medical treatment for GERD is begun, it typically will need to be continued indefinitely—even though for some patients with only intermittent symptoms of reflux and no esophagitis as such, the medical treatment can be intermittent and performed only during symptomatic episodes.

### Native Human Defense Mechanisms Against GERD

It is recognized that the human body has several internal defense mechanisms by which to protect itself from the harmful effects of acid reflux disease.

For example, most refluxing of stomach liquids occurs during the day when the individual tends to stand upright. Given the upright position, a refluxed liquid is more likely to flow back down into the stomach owing to the effect of gravity.

In addition, so long as the individual is awake, he (or she) will repeatedly swallow whatever fluids are present in the mouth, regardless of whether or not there has been any reflux of stomach liquids. Thus, each swallow will carry any refluxed liquid in the mouth back into the stomach as the consequence of mechanical swallowing.

Equally important, the salivary glands adjacent the oral cavity naturally produce saliva, which contains bicarbonate. Thus, as a consequence of each swallow, bicarbonate-containing saliva travels down the cellular lining of the esophagus. In this manner, the bicarbonate in the migrating saliva neutralizes the relatively small quantity of acid that typically remains in the esophagus, after swallowing and gravity have removed most of the refluxed liquid.

Accordingly, gravity, mechanical swallowing, and the bicarbonate in saliva are important protective mechanisms for the esophagus—but unfortunately, these mechanisms are effective only when the individual is in an upright position. At night and during sleep, gravity is not in effect; swallowing stops; and the secretion of saliva is reduced. Consequently, any regurgitation or reflux that occurs at night is more likely to result in acid remaining in the esophagus for a much longer duration and to cause far greater damage to the esophagus.

### C. Recognized Causes of Heartburn & GERD

As demonstrated by the range and variety of pertinent medical and scientific publications, the recognized causes of GERD are usually multiple, often complex, and typically vary for different individuals, or sometimes even for the same individual on alternative occasions.

Today, it is medically recognized that for a relatively small number of patients afflicted with GERD, these persons produce abnormally large amounts of stomach acid—but this is an uncommon cause and is not seen as a major contributory factor for the vast majority of GERD patients. Instead, the primary factors that meaningfully contribute to the occurrence of GERD as a diagnosed pathological condition are: (i) the lower esophageal sphincter muscle; (ii) hiatial hernias; (iii) esophageal contractions; (iv) emptying of the stomach; and (iv) antibiotic and anti-inflammatory medications. Each of these is reviewed below.

### The Lower Esophageal Sphincter Muscle

The action of the lower esophageal sphincter (“LES”) muscle is probably the most important factor or mechanism for preventing acid reflux disease. As illustrated by FIG. 1 herein, anatomically, the esophagus is a muscular tube that extends from the lower throat to the stomach. The LES is a specialized ring of muscle that surrounds the lower-most end of the esophagus where it joins the stomach. The musculature of the LES is active most of the time; and is contracting and closing off the passage from the esophagus into the stomach. The closing of the passage in the normal stomach prevents reflux. When food or saliva is swallowed, the LES muscle relaxes for a few seconds to allow the food or saliva to pass from the esophagus into the stomach, and then it closes again.

Several different abnormalities of the LES have been found in patients suffering from GERD. All of these, however, result in a failure of the LES to close properly, a condition illustrated by FIGS. 2A and 2B respectively.

A first kind of abnormality is a weak contraction of the LES muscle, which results in a partially open passageway and reduces the ability of the LES to prevent regurgitation.

A second kind of abnormality is an unwanted relaxation of the LES muscle, a condition termed “transient LES relaxations”. These relaxations are abnormal in that they do not accompany swallows and they last for a relatively long time, up to several minutes in duration. These prolonged relaxations allow reflux to occur more easily. The transient LES relaxations occur in patients with GERD most commonly after meals when the stomach is distended with food.

A third more recently-described abnormality in patients with GERD is a muscular laxity of the LES. Specifically, distending pressures open the LES more in patients with GERD than in individuals not suffering from GERD. This defect results an easier opening of the LES and a greater upward flow of acid from the stomach into the esophagus.

### Hiatial Hernia

Hiatial hernias contribute to acid reflux, although the way in which they contribute is still not clear. It is recognized that a majority of patients with GERD will also have hiatal hernias, but many other GERD patients apparently do not. For this reason, it is no longer considered necessary today for a human to have a hiatial hernia in order to be diagnosed with GERD. Equally important, many people have hiatal hernias as such, but concurrently do not show any symptoms of acid reflux disease.

Anatomically, the normal condition illustrated by FIG. 1. As seen therein, the LES muscle is located at the same level where the esophagus passes from the chest through the diaphragm and into the abdomen. Then, when there a hiatal hernia exists, a small part of the upper stomach that attaches to the esophagus is pushed up through the diaphragm. As a result, a small part of the stomach and the LES muscle come to lie in the chest cavity; and the LES muscle no longer lies at the level of the diaphragm.

It appears that the diaphragm that surrounds the LES muscle is important in preventing acid reflux. That is, in individuals without hiatal hernias, the diaphragm surrounding the esophagus is continuously contracted, but then relaxes with mouth swallowing. Thus, the barrier against refluxing is a force equal to the sum of the pressures generated by the LES muscle and the diaphragm in combination. When the LES muscle moves into the chest cavity (as with a hiatal hernia), the diaphragm and the LES muscle continue to exert their pressures and barrier effect. However, they now do so at locations that differ from the normal; and the pressures generated by the LES musculature and the diaphragm is no longer additive. Instead, a single, high-pressure barrier to reflux is replaced by two barriers of lower pressure, and reflux thus occurs more easily.

### Esophageal Contractions

As previously described above, mouth swallowing causes a ring-like wave of contraction of the esophageal muscles, which narrows the lumen of the esophagus. The esophageal contractions (commonly referred to as “peristalsis”) begin in the upper esophagus and then travel to the lower esophagus. These contractions repeatedly push food, saliva, and whatever else lies within the esophagus into the stomach.

However, if and when the wave of contraction is defective, the refluxed acid is not pushed back into the stomach. Note that in patients afflicted with GERD, several abnormalities of contraction have been described. For example, waves of contraction may not begin after each swallow; or the waves of contraction may end before they reach the stomach. Also, the pressure forces generated by the esophageal contractions may be too weak to push the acid back into the stomach.

Such abnormalities of contraction, which reduce the clearance of acid from the esophagus, are found frequently in patients suffering from GERD. In fact, these abnormalities are found most often in those patients with the most severe instances of GERD. The effects of abnormal esophageal contractions typically are worse at night when the patient lies prone, because gravity is not then able to help return refluxed acid in the esophagus to the stomach.

### Emptying of the Stomach

It is generally recognized that most acid reflux occurs during the daytime hours, and commonly occurs after the eating of a meal. Such occurrences of acid reflux are believed to be due to transient lower esophageal sphincter (“LES”) relaxations, caused by distention of the stomach with food. A minority of patients with GERD (typically about 20%) has been found to have stomachs that empty abnormally slowly after eating a meal. This slower speed for the emptying of the stomach is believed to prolong the distention of the stomach with food after meals; and consequently, the longer time required for emptying of the stomach prolongs that time period during when acid reflux is likely to occur.

### Antibiotic and Anti-Inflammatory Medications

The deleterious effects on the stomach of antibiotic and anti-inflammatory medications are well documented in the published medical literature. Patients using antibiotic and anti-inflammatory medications routinely report difficult heartburn and severe sour stomach symptoms after treatment; and also note in particular the occurrence of such problems after major surgery, where the course of treatment with these antibiotic and anti-inflammatory medications is typically maintained for a significantly long duration of time.

### D. Natural Remedies for Treating Heartburn and GERD

If and when a human subject experiences heartburn symptoms, it is most important that he/she be evaluated by a physician for gastro-esophageal reflux disorder. If not treated properly, GERD may result in serious medical problems including esophagitis (inflammation of the esophagus), stricture (narrowing) of the esophagus, esophageal ulcers (open sores on the lining of the esophagus) and esophageal bleeding.

With regard to the treatment of heartburn symptoms and/or GERD, a number of natural remedies are known, some of which are regarded today as merely folk medicines. Some of the most commonly used forms of natural treatment are summarily described below.

### Dietary Changes to Avoid Heartburn Symptoms

A number of different foods and beverages are known that commonly act as triggers for heartburn. Removing the foodstuffs and liquids listed by Table B below from the normal diet has served to avoid heartburn and symptoms of GERD.

TABLE B

| | |
|---|---|
| Citrus fruits; | Fatty Foods; |
| Spicy foods; | Coffee; |
| Citrus juice; | Carbonated beverages; |
| Alcoholic beverages | Chocolate; |
| Peppermint; | Spearmint; |
| Tomatoes; | Raw onions; |
| Garlic; and | Vinegar. |

In addition, it is strongly recommended that the person eat his last meal at least two to three hours before lying down; that he eat smaller portion sizes than before; and that he take adequate time to eat slowly and to chew the food completely.

### Reduction of Stress

Many persons live a hectic lifestyle that contributes to their heartburn symptoms and GERD; and about 52 percent of such persons believe that work-related stress makes their GERD problems worse. Curiously however, although some people report that severe stress makes their heartburn symptoms worse, there is not as yet an established direct linkage between heartburn and stress.

Nevertheless, it is indisputable that stress can disrupt our normal living routines; and compels us to eat the wrong foods, or smoke, or drink excessive quantities of coffee or alcohol—all of which tend to trigger heartburn. Stress also slows down the emptying of the stomach, which also increases the likelihood of heartburn.

For these reasons, many persons advocate using one or more natural means for reducing stress. Among the natural methods that commonly are used to manage human stress are: spiritual meditation; physical relaxation techniques; and anxiety control training.

### Deglycyrrhizinated Licorice

Deglycyrrhizinated Licorice (“DGL”) is a folk remedy used for alleviating heartburn. It is a form of the herb licorice that has had the glycyrrhizin component removed to reduce the risk of glycyrrhizin-related side effects such as high blood pressure and water retention. Although some research studies suggest licorice may decrease inflammation, inhibit the growth of potentially harmful stomach bacteria, and help with ulcers; to date, there have not been any formally conducted clinical trials on the use of licorice for heartburn or GERD.

### Aloe Vera Juice

The juice from the aloe vera plant is another natural home remedy that is used to soothe an irritated esophagus. Although there is no reported scientific evidence, to evidence its effectiveness, aloe vera juice has a long history of use in Europe as a natural home remedy to relieve heartburn.

Typically, approximately ¼ cup of aloe vera juice is taken (by adults) approximately 20 minutes before a meal. The aloe vera should not contain any aloe latex, aloin, or aloe-emoin compounds—substances present in the aloe plant that are very powerful laxatives. Also aloe gel is not to be taken directly from the plant as a remedy, as the gel can be contaminated with the latex. Instead, only gel or juice preparations specifically made for internal use by humans should be employed.

### Slippery Elm Herb

Slippery Elm is an herb that was once a popular drugstore remedy for sore throats in North America. The herb was listed in the United States Pharmacopeia, a formal compendium of drug standards, until 1960. The herb is a member of the elm family, the slippery elm tree (Ulmus rubra muhl) that grows primarily in the eastern regions of North America.

The Slippery Elm herb is a long known folk remedy that has not as yet been scientifically evaluated as a treatment for heartburn symptoms and GERD. The inner bark of the Slippery Elm tree contains mucilage, a gel-like substance that swells when it is mixed with water. This Slippery Elm mucilage is used to coat the esophagus and reduce irritation.

In addition, Slippery Elm herb is often the primary ingredient in herbal sore throat lozenges; and can be found either in health food stores or in the natural food section of some grocery stores and drug stores.

### Marshmallow Herb

The herb Marshmallow (Althaea officinalis) contains mucilage, which is believed to be effective to coat and soothe the lining of the esophagus. It is another folk remedy that is used for heartburn.

Herbalists often recommend marshmallow root tea. It is usually made by adding one tablespoon of the dried root to a cup (8 oz.) of boiling water; steeping it covered for at least 10 minutes; and then straining the liquid. Herbalists usually suggest drinking up to three cups a day.

### E. Pharmaceutical Drugs & Pharmacological Formulations

A wide variety of different pharmaceutical preparations have been formulated and used to treat heartburn symptoms and gastro-esophageal reflux disorder. Of these, the majority of Americans has become acquainted with antacids, which functions in-situ by reducing the acidity of the stomach contents and today is the least expensive pharmacological treatment for heartburn.

Almost as well known are H2-receptor antagonists (“H2 blockers”) and Proton Pump Inhibitors (“PPIs”). The Proton Pump Inhibitors cost a little more than antacids, but are generally more convenient for use; and some formulations of H2 blockers can be conveniently purchased as over the counter drugs (i.e., without a physician's prescription. PPIs are seen to be more effective than either antacids or H2 blockers, but have major side effects and are far more costly. In severe cases, physicians may favor combining different kinds of drugs, such as concurrent administrations of antacids and H2 blockers, or combinations of PPIs and prokinetic drugs. However, PPIs without additional medications are generally preferable to their combination.

A summary review of these conventionally known pharmaceutical formulations as remedies for heartburn and GERD is presented below.

### Antacids

Antacids are inexpensive, over-the-counter remedies, which neutralize digestive acids in the stomach and esophagus—at least in mild instances of heartburn. While many people find tablets more convenient, liquids actually provide faster relief because the tablets must be chewed thoroughly in order to be effective. Generally, the best time to take an antacid is immediately after a meal or when symptoms occur.

There are three basic salts used in antacid formulations: magnesium, aluminum, and calcium. Some physicians consider magnesium-based antacids and aluminum-based antacids (including Di-Gel, Maalox, and Mylanta) to be the most cost-effective heartburn drugs. A major side effect of magnesium hydroxide is diarrhea, while the most common side effect of antacids containing aluminum hydroxide is constipation.

In comparison, antacid formulations which are particularly high in calcium (e.g., “Tums”, “Rolaids”, “Titralac”, and “Atka-2”) are probably the strongest. Note also that calcium carbonate products have been used as antacids for centuries in the form of chalk powder and oyster shell. They, too, can be constipating if consumed in sufficient quantities.

Sodium bicarbonate, or baking soda, which is less powerful than other antacid formulations, is the active ingredient in many seltzer antacids (e.g., “Alka-Seltzer”, “Bromo-Seltzer”) and is intrinsically present in mineral water.

Similarly, because no single chemical agent is perfect for use, many antacid formulations combine several types of ingredients to balance their respective side effects. Thus, for example, the “Maalox” formulation combines magnesium and aluminum; and the “Gaviscon” formulation combines antacids with alginic acid, a substance derived from marine algae.

### Proton Pump Inhibitors

Proton Pump Inhibitors (“PPIs) are effective at lowering the production of gastric acid. PPIs function in-situ by inactivating a specific enzyme responsible for the final step of acid release in the stomach. These compositions can reduce gastric acid secretion by more than 95% without causing systemic side effects.

Initially, PPIs were only available by prescription. In 2003, omeprazole (or “Prilosec”) became the first pharmaceutical compound to become available without a prescription; and it also is the only one approved by the FDA for repeated courses of treatment for erosive esophagitis.

Today, PPIs now available by prescription include: lansoprazole (“Prevacid”), rabeprazole (“AcipHex”), pantoprazole (“Protonix”), omeprazole (“Zegerid”), and esomeprazole (“Nexium”). Note also that omeprazole is an immediate-release medication, in contrast to all the others which are delayed-release drugs.

PPIs are also the drugs of choice for erosive esophagitis. This pathological condition usually recurs when the drug is stopped; thus, a long-term treatment is usually necessary when using such drugs. All these medications are very effective in their ability to heal esophagitis and alleviate heartburn symptoms.

Lastly, although they have numerous advantages, PPIs are quite expensive. Moreover, PPIs tend to make the human gastrointestinal tract more susceptible to bacterial infections. Despite these concerns, however, PPIs are today the most preferred pharmaceutical preparation used for reflux esophagitis and for patients with unremitting GERD-derived respiratory symptoms. Also, PPIs are often tried first for frequent, uncomplicated heartburn; but, once the symptoms are controlled, a less expensive medication (such as an H2-receptor antagonist) is often substituted by the physician.

### Histamine H2-Receptor Antagonists

For chronic reflux disease, histamine H2-receptor antagonists or “H2 blockers” are now widely used. H2 blockers are commonly available either by prescription; or, in smaller dose quantities, are sold freely over the counter. H2 blockers are often effective for treating GERD symptoms that don't respond to antacids or changes in eating habits.

H2 blockers function in-vivo by countering the effect of histamine (which stimulates gastric acid); and thereby act to decrease the amount of acid that the stomach produces. These drugs act directly on the stomach's acid-secreting cells to stop them from making hydrochloric acid, particularly at night when acid gathers in the stomach and can wash upwards into the esophagus. A listing of the more commonly available histamine H2-receptor antagonists is presented by Table C below.

TABLE C

| Generic name | Brand name | Use Side effects/Comments |
|---|---|---|
| Cimetidine | Tagamet | Relieves heartburn and functional |
| | | Dyspepsia pain and promotes ulcer |
| | | healing by decreasing stomach acid |
| | | Rarely may cause diarrhea, |
| | | constipation, dizziness, anxiety, |
| | | depression, drowsiness, sleeplessness, |
| | | headache, irregular heartbeat, |
| | | increased sweating, burning, itching, |
| | | redness of skin, fever, confusion in ill or |
| | | elderly people. May interfere with the |
| | | absorption of anticoagulants, |
| | | antidepressants, and hypertension |
| | | medications. |
| famotidine | Pepcid | Same as above. No serious drug |
| | | interactions known. |
| nizatidine | Axid | |
| ranitidine | Zantac | Same as above. At high doses may |
| | | interact with anticoagulant. |

Historically, cimetidine (“Tagamet”) was the first H2 blocker freely sold to the public. Others H2 blockers now available in the United States include ranitidine (“Zantac”), famotidine (“Pepcid”), and nizatidine (“Axid”). However, all H2 blockers are recognized as being equally effective; and thus switching to another brand or formulation (if one fails to work) is likely to be fruitless. In comparison, actually increasing the dosage often can be beneficial.

In general, H2 blockers are considered to be relatively safe for regular use. Nevertheless, H2 blockers can produce some undesirable side effects, and therefore care must be exercised when taking such medication.

### Prokinetic Agents

Prokinetics, or gastrokinetics as occasionally called, are a wide-ranging category of drugs that help empty the stomach of acids and fluids. Prokinetic agents can also improve lower esophageal sphincter (“LES”) muscle tone. These pharmaceutical preparations are used only for occasional cases of GERD, either with or in place of H2 blockers; and particularly when the stomach appears to empty slowly.

Particular risk is associated with the use of Prokinetic agents. It is noted that cisapride (“Propulsid”) was recalled from the U.S. market in 2000 after it was linked to more than 300 reports of heart rhythm abnormalities, which included more than 80 deaths. Its predecessor compounds, metoclopramide (“Reglan”) and bethanechol (“Urecholine”), remain FDA approved and available by prescription, but have a wide variety of side effects.

For more information and medical details about these different pharmaceutical formulations, please see the following scientific publications, all of which are merely representative examples: Johnson, D A, Benjamin, S B, Vakil, N B, et al. Esomeprazole once daily for 6 months is effective therapy for maintaining healed erosive esophagitis and for controlling gastroesophageal reflux disease symptoms: A randomized, double-blind, placebo-controlled study of efficacy and safety. Am J

Gastroenterol 2001; 96: 27-34. Sandmark, S, Carlsson, R, Fausa, 0, et al. Omeprazole or ranitidine in the short-term treatment of ulcerative reflux oesophagitis. Results of a double-blind randomized Scandinavian multicenter study. Scand J Gastroenterol 1988; 23: 625-632; Antonson, C W, Robinson, M G, Hawkins, T M, et al. High doses of histamine antagonists do not prevent relapses of peptic esophagitis following therapy with a proton pump inhibitor. Gastroenterology 1990; 98: A16; Bank, S, Greenberg, R. Alternate day omeprazole in H2 receptor-antagonist resistant reflux esophagitis. Gastroenterology 1991; 100: A29; Dent, J, Yeomans, N D, Mackinnon, M, et al. Omeprazole v randitidine for prevention of relapse in reflux oesophagitis. A controlled double blind trial of their efficacy and safety. Gut 1994; 35: 590-598; Hallerback, B, Unge, P, Carling, L, et al. Omeprazole or ranitidine in long-term treatment of reflux esophagitis. Gastroenterology 1994; 107: 1305-1311; Ferguson, R, Dronfield, M W, Atkinson, M. Cimetidine in treatment of reflux oesophagitis with peptic stricture. Br Med J 1979; 2: 472-474; Marks, R D, Richter, J E, Rizzo, J, et al. Omeprazole versus H2-receptor antagonists in treating patients with peptic stricture and esophagitis. Gastroenterology 1994; 106: 907-915; Swarbrick, E R, Gough, A L, Foster, C S, et al. Prevention of recurrence of oesophageal stricture: A comparative study of lansoprazole and high dose ranitidine. Eur J Gastroenterol Hepatol 1996; 8: 431-438; Klinkenberg-Knol, E C, Festen, H P M, Jansen, J B M J, et al. Long-term treatment with omeprazole for refractory reflux esophagitis: Efficacy and safety. Ann Intern Med 1994; 121: 161-167; Neumann, C S, Iqbal, T H, Cooper, B T. Long term continuous omeprazole treatment of patients with Barrett's oesophagus. Aliment Pharmacol Ther 1995; 9: 451-454; Venables, T L, Newland, R D, Patel, A C, et al. Omeprazole 10 milligrams once daily, omeprazole 20 milligrams once daily or ranitidine 150 milligrams twice daily, evaluated as initial therapy for the relief of symptoms of gastro-oesophageal reflux disease in general practice. Scand J Gastroenterol 1997; 32: 965-973; Richter, J E, Campbell, D R, Kahrilas, P J, et al. Lansoprazole compared with ranitidine for the treatment of nonerosive gastroesophageal reflux disease. Arch Intern Med 2000; 160: 1803-1809.

### F. Other Relevant Developments and Innovations Described in the Published Literature

Within the patent and scientific literature, a number of different innovative approaches and investigations have been pursued, some of which are more relevant and material to the treatment of heartburn and GERD than others. Nevertheless, for general knowledge and overall awareness, the following representative examples will serve to demonstrate the true range and diversity of conventionally known information:

### Conventionally Known Uses for Apple Cider Vinegar

A few issued U.S. patents and published U.S. patent applications concern themselves with uses for apple cider vinegar. Merely illustrative of these are the following:

U.S. Patent Application Publication 2008/0207754 describes a throat rinse delivery system for preventing or reducing acid reflux or GERD by delivering a packaged dose of vinegar directly to the back of the throat without exposing the taste buds, and which is immediately followed by a packaged dose of water or other pleasant tasting chaser liquid to rinse the throat. As stated therein, extensive anecdotal evidence and patient testimonials indicate that apple cider vinegar provides temporary relief from the symptoms of GERD; but that the ingestion of vinegar is difficult, if not impossible for most people because vinegar has a notoriously unpalatable taste and smell. The innovative is thus an applicator and delivery system, which generally comprises a joined pair of sealed flexible pouches or receptacles—the first of which is filled with undiluted apple cider vinegar while the second is filled with a liquid chaser, usually distilled water. In delivery and use, the vinegar receptacle is lifted to the mouth and burst open such that the vinegar is poured into the throat, and is followed immediately by the second receptacle and the rapid pouring out of the water chaser to prevent reflex gagging.

U.S. Pat. No. 6,063,364 describes a toothpaste for cleaning the teeth and for combating disagreeable odors and malodorousness in the human mouth comprising a toothpaste composition and a solution of one part water and three parts apple cider vinegar.

U.S. Pat. No. 5,993,852 discloses a dietary supplement comprising a lyophilized reaction product of sodium bicarbonate and raw apple cider vinegar.

### Conventionally Known Treatments for Heartburn and GERD Symptoms

A variety of issued U.S. patents and published U.S. patent applications are directed to compositions and methods for treating heartburn symptoms and/or GERD. Among the more interesting examples are the following:

U.S. Patent Application Publication 2008/0248136 discloses a composition and method for the simultaneous alleviation of symptoms for both acute and chronic gastric and esophageal reflux disorder. The composition comprises specifically limited quantities of limonene and at least one antacid selected from the group consisting of calcium carbonate, aluminum hydroxide, and magnesium hydroxide. The method orally administers the formulated composition to a mammal.

U.S. Patent Application Publication 2004/0170696 discloses a composition whose primary use is to relieve occasional heartburn and digestive disorder, including GERD and stomach/intestine complaints. The composition is comprised of honey and raw food fibers; and can be processed into a compressed product.

U.S. Pat. No. 7,501,400 describes a method for decreasing the production or secretion of gastric acid in a human subject via the administration of an oligonucleotide which consists of 12-30 nucleobases and is entirely complimentary to a region of an RNA transcript encoding an alpha chain of the human proton pump.

U.S. Pat. No. 7,501,135 discloses a method of reducing gastric acid secretion in animals and humans via the administration of an extract of a plant of the genus Hoodia or Trichocaulon.

U.S. Pat. No. 7,498,337 provides a method of treating peptic ulcer, Zollinger-Ellison syndrome, reflux esophagitis, or symptomatic gastroesophageal reflux disease via the administration of a novel chemically defined organic compound.

U.S. Pat. No. 5,989,588 presents a method for preventing heartburn in a patient by administering, at a time following a heartburn inducing event but prior to actual development of heartburn, a H2 antagonist and an antacid having a specified acid neutralizing capacity.

U.S. Pat. No. 5,854,267 describes a method for preventing heartburn in a susceptible patient following ingestion of a heartburn-inducing food or beverage via the administration of famotidine 30 minutes prior to consumption of that food or beverage by the patient.

U.S. Pat. No. 5,667,794 provides a method for treating a human suffering from heartburn but having no substantial esophageal erosion via the administration of famotidine in the absence of any antacid.

U.S. Pat. No. 5,229,137 discloses a method of providing immediate and sustained relief from an episode of heartburn by orally administering an antacid and an H2 receptor antagonist concurrently to the human patient.

### Conventionally Known Uses for Honey

A variety of published U.S. patent applications are directed to the use of honey as an ingredient in medical dressings, particularly for topical wound dressings. These developments are merely illustrated and exemplified by the following:

U.S. Patent Application Publication 2004/0054313 which discloses a medical composition comprising a combination of honeys with a viscosity increasing agent.

U.S. Patent Application Publication 2003/30136274 which provides an applicator for applying a honey composition topically to a site, in which the applicator includes a sealable reservoir, a pressure assembly, and a nozzle assembly—all in fluid communication with each other.

U.S. Patent Application Publication 2004/0127826 which describes a method of manufacturing a honey based dressing, in which the honey effects a change in the physical characteristics of the dressing, and is useful as a moist application.

U.S. Patent Application Publication 2009/0012440 which reveals a wound dressing as a substantially solid sheet having three distinct layers, in which only the first uppermost layer comprises honey and a gelling agent.

U.S. Patent Application Publication 2005/0033213 which discloses a three layered contact wound dressing which has honey in its wound-contacting and intermediate layers.

In addition, a variety of published research is directed to the use of honey as an ingredient in the treatment of peptic ulcers and gastroenteritis. These uses are merely illustrated and exemplified by the following:

### Concerning Peptic Ulcers

Kandil, A.; El-Banby, M.; Abdel-Wahed, K.; Abdel-Gawwad, M.; Fayez, M. (1987) Curative properties of true floral and false nonfloral honeys on induced gastric ulcer. Journal of Drug Research (Cairo) 17 (1-2): 103-106; Kandil, A.; El-Banby, M.; Abdel-Wahed, K.; Abdel-Gawwad, M.; Fayez, M. (1989). “Curative properties of floral honey and honey from sugar-fed bees on induced gastric ulcers.” Fourth International Conference on Apiculture in Tropical Climates, Cairo, International Bee Research Association, London. 68-69; Ali, A. T. M. M.; Al-Humayyd, M. S.; Madan, B. R. (1990) Natural honey prevents indomethacin- and ethanol-induced gastric lesions in rats. Saudi Medical Journal 11 (4): 275-279; Ali, A. T. M. M.; Chowdhury, M. N. H.; Al Humayyd, M. S. (1991) Inhibitory effect of natural honey on Helicobacter pylori. Tropical Gastroenterology 12 (3): 139-143; Ali, A. T. M. M. (1991) Prevention of ethanol-induced gastric lesions in rats by natural honey, and its possible mechanism of action. Scandinavian Journal of Gastroenterology 26: 281-288; Al Somai, N.; Coley, K. E.; Molan, P. C.; Hancock, B. M. (1994) Susceptibility of Helicobacter pylori to the antibacterial activity of manuka honey. Journal of the Royal Society of Medicine 87 (1): 9-12; Ali, A. T. M. (1995) Natural honey accelerates healing of indomethacin-induced antral ulcers in rats. Saudi Medical Journal 16 (2): 161-166; Ali, A. T. M. M. (1995) Natural honey exerts its protective effects against ethanol-induced gastric lesions in rats by preventing depletion of glandular nonprotein sulfhydryls. Tropical Gastroenterology 16 (1): 18-26; Al-Swayeh, O. A.; Ali, A. T. M. (1998) Effect of ablation of capsaicin-sensitive neurons on gastric protection by honey and sucralfate. Hepato-Gastroenterology 45 (19): 297-302.

### Concerning Gastroenteritis

Salem, S. N. (1981) Honey regimen in gastrointestinal disorders. Bulletin of Islamic Medicine 1: 358-362; Haffejee, I. E.; Moosa, A. (1985) Honey in the treatment of infantile gastroenteritis. British Medical Journal 290: 1866-1867; Linnett, P. (1996). Honey for equine diarrhoea. Control and Therapy: 906.

### G. Overview & Perspectives

As evidenced by the foregoing, a range of diverse compositions of matter and a variety of different techniques have been developed and utilized for the treatment of heartburn and for mediating the symptoms of GERD. Some of these are demonstrably useful; others, however, are unfortunately at best ineffective and at worst act merely to aggravate the underlying pathological condition. Equally important, even the most effective treatments employed to date routinely employ chemically synthesized pharmaceutical formulations as the compositions of choice—all of which are known as being limited in acceptable dosage quantity, and become less tolerated by the human body over extended time, and also cause undesirable side effects for the user. Consequently, all of the conventionally available compositions and treatments employed to date are far less than optimal medicinal regimens, and frequently are short-term treatments of severely limited duration and effect.

Accordingly, there remains a long standing and well recognized need for a formulated medicament which is prepared as a fluid blending of ingredients which exist in nature, and yet is effective as either a preventative or a remedial method of treating heartburn and the symptoms of GERD. Were such a medicinal composition and treatment methodology to be developed, persons of ordinary skill in this medical filed would find such an innovation to be an unexpected advance and a major unforeseen benefit to persons suffering from heartburn and/GERD symptoms.

### SUMMARY OF THE INVENTION

The present invention has multiple aspects, which are summarized as follows.

A first aspect is a medicament to be orally ingested by a living human subject for the treatment of a heartburn inducing event or an acid reflux episode, said medicament comprising a fluid blending of:

a concentrated vinegar;

an undiluted bioactive honey which has substantial non-peroxide antibacterial activity and retains its non-peroxide antibacterial activity after being combined with said concentrated vinegar;

at least one flavoring agent; and

at least one additional sweetener,

whereby said medicament

(i) has a determinable pH value ranging from about 2.5 to 6.0,

(ii) demonstrates non-peroxide antibacterial activity in-situ, and

(iii) is sufficiently palatable to the human mouth such that said medicament can be swallowed by a human subject without substantial gagging.

A second aspect of the invention is a method for treating a heartburn-inducing event in a living human subject, said treatment method comprising the steps of:

obtaining a medicament comprising a fluid blending of a concentrated vinegar; an undiluted bioactive honey, which has substantial non-peroxide antibacterial activity and retains demonstrable non-peroxide antibacterial activity after being combined with said concentrated vinegar; at least one flavoring agent; and at least one additional sweetener,

whereby said medicament
(i) has a determinable pH value ranging from about 2.5 to 6.0,
(ii) demonstrates non-peroxide antibacterial activity in-situ, and
(iii) is sufficiently palatable to the human mouth such that said medicament can be swallowed by a human subject without substantial gagging;

orally ingesting an effective quantity of said medicament on at least one occasion;

allowing said orally ingested medicament to react with the stomach contents of the living human subject such that
(a) said ingested medicament causes and maintains a milder acidic pH value for the stomach of the human subject, and
(b) said ingested medicament exerts non-peroxide antibacterial activity within the esophagus and stomach of the human subject;
   and

determining that the severity of the heartburn-inducing event has become markedly reduced.

A third aspect of the invention is a method for treating an acid reflux episode in a living human subject, said treatment method comprising the steps of:

obtaining a medicament comprising a fluid blending of
a concentrated vinegar;
an undiluted bioactive honey, which has substantial non-peroxide antibacterial activity and retains demonstrable non-peroxide antibacterial activity after being combined with said concentrated vinegar;
at least one flavoring agent; and
at least one additional sweetener,
whereby said medicament
(i) has a determinable pH value ranging from about 2.5 to 6.0,
(ii) demonstrates a marked non-peroxide antibacterial activity in-situ, and
(iii) is sufficiently palatable to the human mouth such that said medicament can be swallowed by a human subject without substantial gagging;

orally ingesting an effective quantity of said medicament on at least one occasion;

allowing said orally ingested medicament to react with the stomach contents of the living human subject such that
(a) said ingested medicament causes and maintains a milder acidic pH value for the stomach of the human subject,
(b) said ingested medicament exerts non-peroxide antibacterial activity within the esophagus and stomach of the human subject; and

determining that the severity of the acid reflux episode has become markedly reduced.

### BRIEF DESCRIPTION OF THE FIGURES

The present invention may be more easily understood and more readily appreciated when taken in conjunction with the accompanying Drawing, in which:

FIG. 1 illustrates the anatomy of the normal condition for the lower esophageal sphincter (“LES”) muscle to open and close properly; and

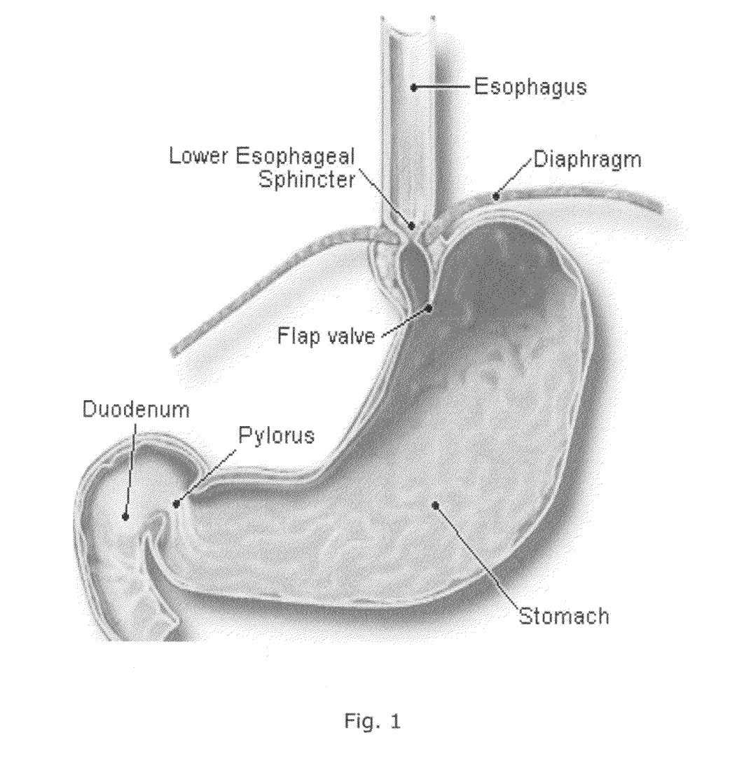

FIGS. 2A and 2B illustrate the anatomy of the abnormal condition where there is a failure of the lower esophageal sphincter (“LES”) muscle to close properly.

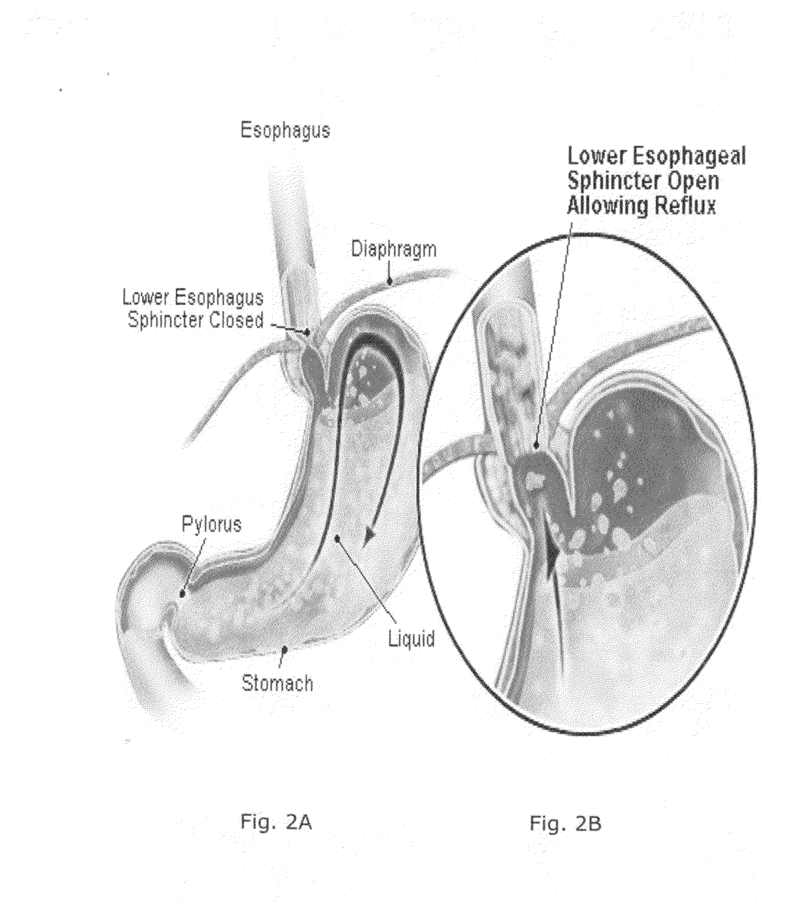

### DETAILED DESCRIPTION OF THE INVENTION

The present invention is a medicament which can be prepared as an all-natural, or natural, or organic, or artificial formulation formed primarily of synthetic substances; and is a medicinal fluid blending of ingredients which is to be orally ingested by a living human subject for the prophylactic or therapeutic treatment of a heartburn-inducing event or an acid reflux episode (GERD).

In its preferred embodiments, the complete medicament is a fluid blending of at least one concentrated vinegar made by the fermentation of a fruit sugar, or a vegetable, or a grain; an undiluted bioactive honey having unique, non-peroxide antibacterial activity; a natural flavoring agent or combination of different natural flavors to neutralize the taste of the concentrated vinegar; and a natural sweetener to give the fluid blending a palatable taste. However, in each instance regardless of particular formulation, the concentrated vinegar of the medicament serves to treat the symptoms of the heartburn and/or GERD; and the undiluted bioactive honey of the medicament provides unique non-peroxide antibacterial activity to treat inflammation of the esophagus and infections of the stomach.

### DEFINITIONS

Although many of the words, terms and titles employed herein are commonly employed and conventionally understood in their traditional usage and context by persons ordinarily skilled in this art, a short listing of definitions is presented below in order to provide a minimal vocabulary; and as an aid and guide for avoiding misinformation, misunderstandings, and ambiguities in terminology which often exist in this technical field; and to introduce specialized terms and jargon for recognizing the particulars of the present invention and for appreciating the true scope and breadth of the claims recited below.

Medicament: Any substance, formulation, composition, or preparation used for medical treatment of a living human subject either in advance to prevent or remedially to counteract a pathological state, or a disease, or a disorder.

All-Natural medicament: Any medicinal substance, formulation, composition, or preparation that contains no artificial compounds or chemically synthesized ingredients and is useful as a medical treatment.

Natural medicament: Any medicinal substance, formulation, composition, or preparation comprised primarily of ingredients that exist in or are created by nature, but which includes one or more additional compounds, enhancements, fractions, or chemically synthesized materials in small proportional ratio quantities totaling less than about 25% of the matter.

Organic medicament: Any substance, formulation, composition, or preparation that exists in the state or form created by nature and has been obtained without the use of either artificial methods or chemically synthesized compositions.

Concentrated vinegar: An aqueous solution of acetic acid ranging in strength from about 2.5 to about 6.0% (w/v).

Vinegar grain strength: A mathematical parameter of vinegar which indicates its acetic acid content (w/v); and in which the grain strength of a vinegar is always calculated as being ten times the acetic acid content then present in that particular vinegar. For example, there are 50, 100, and 200 grain strength vinegars; and accordingly, 50 grain strength vinegar contains 5% acetic acid (w/v), 100 grain strength vinegar contains 10% acetic acid (w/v), and 200 grain strength vinegar contains 20% acetic acid (w/v).

Bioactive honey: A kind or source of honey having a recognized substantial antibacterial activity in-situ which is not destroyed by the enzyme catalase, and is unaffected by the presence or absence of hydrogen peroxide, and is not neutralized in activity by the addition of diluting organic acids.

Non-peroxide antibacterial activity: A substance having a demonstrable antibacterial activity in-situ which is not destroyed by the enzyme catalase and whose antibacterial activity is not dependent upon the production or presence of hydrogen peroxide.

Natural flavoring agent: Any flavored substance, formulation, composition, or preparation that exists in or is created by nature, contains no artificial compounds or chemically synthesized ingredients, and is able provide a distinctive sensation of smell and taste to food or drink.

Natural additional sweetener: Any sugar-like substance, formulation, composition, or preparation that exists in or is created by nature, contains no artificial compounds or chemically synthesized ingredients, and can be used to sweeten food or drink.

pH value: The logarithm, to the base 10, of the reciprocal of the concentration of hydrogen ions in an aqueous based liquid; and is a convenient means of expressing small differences in the acidity or alkalinity among aqueous based fluids.

UMF® (or “Unique Manuka Factor”): A numbering system, also known as the UMR® rating system, which is used generally with different kinds and sources of honey as a comparative measurement or assessment of antibacterial activity, and is based on the known antibacterial properties of the common antiseptic, phenol. Thus for example, a type of honey that has a UMF® value of 10 is said to have the same degree of antibacterial activity as a 10% (w/v) solution of phenol.

MGO™ Certified Manuka Honey: An alternative system for certifying what is the minimal content (in mg/kg units) of dietary Methylglyoxal then present in the various kinds and different sources of honey which demonstrably show a non-peroxide antibacterial activity. Thus, a certification of MGO™ 100+ honey means that at least one hundred milligrams per kilogram of dietary Methylglyoxal in that honey; and a certification of MGO™ 550+Manuka Honey means that at least 550 milligrams per kilogram of Methglyoxal exists in that particular Manuka honey.

### I. The Ingredients Comprising the Medicament

The medicament of the present invention is a uniquely formulated medicinal composition of matter suitable for oral ingestion by a living human subject on-demand or in accordance with a time scheduled treatment regimen; and can be prepared in the alternative as an all-natural medicament, or as a natural medicament, or as an organic medicament, or as an artificial medicament formed primarily of synthesized substances. Any of these alternative medicament formats can be beneficially employed for the treatment of a heartburn inducing event or an acid reflux episode.

In each instance and individual format, however, the medicament will comprise a fluid blending of four ingredients, which are:

(A). At least one concentrated vinegar;

(B). At least one undiluted bioactive honey which has substantial non-peroxide antibacterial activity in the undiluted state and retains its non-peroxide antibacterial activity after being diluted with said concentrated vinegar;

(C). At least one flavoring agent; and

(D). At least one additional sweetener.

Each of these required ingredients is described in detail below.

### A. At Least One Concentrated Vinegar

The medicament of the instant invention is preferably prepared as an all-natural, or a natural, or an organic fluid blending; and in these preferred instances will comprise at least one concentrated vinegar which has been made by the fermentation of at least one kind of fruit sugar, or a vegetable, or a grain. By definition, a concentrated vinegar is an aqueous solution of acetic acid ranging in strength (concentration) from not less than about 2.5 (w/v) to about 6.0% (w/v).

The concentrated vinegar of choice is preferably the result of the natural alcoholic and acetic fermentation of at least one fruit sugar; and most typically is the fermentation product of a sugar existing naturally in fruit liquids such as apple ciders, grape juices, pineapple juices, pomegranate juice, citrus fruit juices, raspberry and other berry juices, and coconut water/milk juices.

If desired, preferred concentrated vinegars can also be obtained via the fermentation of vegetable carbohydrates and sugars. Typically these concentrated vinegars will be made from tubers (potatoes, yams, tapioca root, etc.), or from legumes (peas, corn, string beans, soybeans, etc.), or from vine vegetables (tomatoes, squash, pumpkins, etc.). As a particular precaution, however, it will be noted that some vegetable vinegars commercially sold today do not contain the minimally required quantity of acetic acid—i.e., they contain less than 2.5% acetic acid (w/v); and thus, such vegetable vinegars are not “concentrated” by definition. No vinegar regardless of source having less than about 2.5% acetic acid (w/v) is ever acceptable for use when preparing the medicament of the present invention.

A distinct third source of preferred concentrated vinegar is via the alcoholic fermentation of one or more grains; and typically such concentrated vinegars are made from grain such as rice, barley, malt, hops, and rye. These grains contain many different kinds of complex carbohydrates and sugars; and typically are a good alternative source of concentrated vinegars having not less than about 2.5% acetic acid (w/v).

It is expressly understood, however, that for purposes of the instant invention, the true source or precise chemical identity of the fruit sugar, or the vegetable carbohydrates and sugars, or the grain carbohydrates and sugars is neither relevant nor material. To the contrary, it is expected that any and all fruits and fruit sugars, and any vegetable source, and any type of grain as such may be the source of origin for the concentrated vinegar; and that frequently a mixture of different fruits and fruit sugars, and/or alternative vegetable sources, and/or various kinds of grains may be employed in combination for this purpose. All such varieties of concentrated vinegars are deemed to be suitable for use in the present invention so long as they provide enough acetic acid as a natural fermentation product to be in the 2.5 to 6.0% acid concentration range.

For these reasons, some preferred concentrated vinegars having acetic acid levels sufficient to prevent microbiotic life are exemplified by: apple cider vinegar (pH 3.13), red wine vinegar (pH 2.64); white wine vinegar (pH 2.88); rice vinegar (pH 2.56); and plum vinegar (pH 2.88)—all these pH values being measured at 20° C.

### A Markedly High Quantitative Ratio of Acetic Acid in the Concentrated Vinegar

As described and exemplified in detail below, a very high proportional ratio of concentrated vinegar is employed as an ingredient in the fluid blending of the medicament. Note however, that for medicaments that are all-natural, or natural, or organic—the requirement explicitly is for a concentrated vinegar obtained by the fermentation of at least one fruit sugar, vegetable, or grain. Thus, to be suitable for use in the fluid blending of these preferred medicaments, three different and distinct conditions must be met by the vinegar:

(a) The vinegar must be in concentrated form, or at least be relatively undiluted, at the time of use.

In this respect, it will be noted that the term “grain strength” is a conventionally known mathematical parameter of vinegar which indicates the acetic acid content; and that major differences exist among 50, 100, and 200 grain strength vinegars. By definition and common parlance in this technical field, the grain strength of a vinegar (or more commonly “grain” vinegar) is ten times its true acetic acid content. Accordingly, 50 grain strength vinegar has 5% acetic acid; 100 grain strength vinegar has 10% acetic acid; and 200 grain strength vinegar has 20% acetic acid.

In addition, it will be recognized and understood that vinegars with a markedly large grain strength value can be diluted (with water or another miscible liquid) prior to that vinegar being used as an ingredient in the formulated medicament. Nevertheless, this prior-to-use dilution need not detract from nor deny the suitability of that particular vinegar for use in the present invention. This fact and result is demonstrated by the following evidence:

50 grain strength apple cider vinegar has 5% acetic acid and a pH of about 3.13 at 20° C.;

40 grain strength apple cider vinegar has 4% acetic acid and a pH of about 3.18 at 20° C.;

30 grain strength apple cider vinegar has 3% acetic acid and a pH of about 3.21 at 20° C.;

50 grain strength apple cider vinegar+20% water=40 grain strength apple cider vinegar; and

50 grain strength apple cider vinegar+40% water=30 grain strength apple cider vinegar.

(b) The vinegar must be a concentrated vinegar having a demonstrable acetic acid content ranging from about 2.5% to about 6.0% (w/v).

In this regard, it is again emphasized that many commercially sold vinegars (particularly for cooking purposes) do not contain the minimally required quantity of acetic acid—i.e., they contain less than 2.5% acetic acid (w/v). All such vinegars regardless of source are not “concentrated” by definition and are not acceptable or suitable for use as an ingredient in the preparation of the complete medicament. No vinegar having less than about 2.5% acetic acid (w/v) is ever to be used with the present invention.

In contrast, concentrated vinegars having an acetic acid content above about 6.0% (w/v) would be acceptable, either in the super-concentrated form, or as a mildly diluted vinegar whose acetic acid concentration is then still 2.5% (w/v) or greater.

(c) The concentrated vinegar is the product of a fruit, vegetable or grain sugar (or carbohydrate) fermentation process.

This requirement allows the fluid blending to be formulated in the alternative as an “all-natural medicament”, or a “natural medicament”, or as a completely “organic medicament”. Any and all of these variant formats can be made at will using these sources of concentrated vinegar.

### High Proportional Ratio Range for the Ingredient

Initially, it is important to recognize properly and appreciate fully a most basic and undisputed fact about concentrated vinegars: It is impossible for any human to drink even a small amount of any concentrated vinegar without immediately causing violent gagging and regurgitation via the esophageal reflex reaction. This gagging event and regurgitation phenomenon is long known and has been verified as being an unequivocal result and established fact. Nevertheless, it is an unqualified requirement that the vinegar employed in the fluid blending of the medicament always be a markedly large proportional ratio and quantity of concentrated vinegar.

In general therefore, not less than about 44% (w/v) and not more than about 97% (w/v) of the fluid blending comprising the medicament will be concentrated vinegar—where the chosen concentrated vinegar then employed has an acetic acid content ranging from about 2.5% to about 6.0% (w/v). This 44%-97% (w/v) proportional ratio range, however, is merely the broadest range deemed to be useful in preparing the medicament. A more desirable proportional ratio range is from about 45% to about 87% concentrated vinegar; and a highly preferred proportional ratio range is from about 61% to about 84% (w/v) concentrated vinegar.

Clearly therefore, in all formulations of the medicament, the concentrated vinegar is the overwhelmingly predominate, if not actual majority ingredient of the complete medicament.

In addition, because the concentrated vinegar can range from about 44%-97% of the formulation, the measurable quantity of acetic acid actually present within the complete medicament will be a minimum of 1.10% of the formulation and be a maximum of 5.82% of the fluid blending. Furthermore, based in part upon this variable quantitative range for acetic acid content, the final pH value of the complete medicament will typically vary from a relatively mild acid pH of about 6.0 (at the minimal acetic acid content) to a strongly acid pH of about 2.59 to 3.76 (at the maximal acetic acid content).

### Concentrated Apple Cider Vinegar

The most preferred example of a concentrated vinegar for use with the present invention is apple cider vinegar, made by the fermentation of apple cider. During this process, the natural sugars in the apple cider are broken down by bacteria and yeast into alcohol, and then into acetic acid. Typically, concentrated apple cider vinegar contains acetic acid and some lactic, citric and malic acids. Unlike white vinegar, apple cider vinegar is a light yellow-brown color; is often sold in unfiltered and unpasteurized form; and usually has a dark cloudy sediment settled at the bottom of the container.

### Conventionally Known Uses

Historically, apple cider vinegar is a long used folk remedy used in highly diluted form that is said to alleviate or cure many different kinds of ailments. The cures are said to include: allergies, sinus infections, acne, high cholesterol, flu, chronic fatigue, Candida infections, sore throats, diabetes, acid reflux, contact dermatitis, arthritis, and gout. In the main, therefore, its popularity rests on its use primarily as a “fat burner” or as part of an alternative diet to restore alkaline-acid balance.

It is also important to note a most basic and undisputed fact about apple cider vinegar: It was previously and remains today impossible for any human to drink even a small amount of any concentrated vinegar without immediately causing violent gagging and regurgitation via the esophageal reflex reaction. For this reason, even when used as a folk medicine, the apple cider vinegar was always either highly diluted with water or another aqueous based liquid, or was intermixed with one or more palatable foods. In either instance, therefore, the true amount of acetic acid actually ingested as a folk medicine was always very dilute; and for this reason became highly questionable as whether or not it had any useful effect.

### Beneficial Value and In-Situ Effects of Concentrated Vinegar in the Present Invention

It will be noted that the concentrated vinegar actually present in the formulated medicament is carefully controlled to constitute not less than about 41% (w/v) and not more than about 97% (w/v) of the formulated fluid blending. The beneficial value and intended in-situ effects of a human consuming such relatively large quantities of undiluted concentrated vinegar are threefold:

First, acetic acid is a far weaker acid that the hydrochloric acid found in the human stomach during instances of heartburn and GERD, where the typical pH value of the stomach contents is then about 2.0 to 3.0. The introduction of concentrated vinegar (and acetic acid) will actually raise the pH value of the stomach to a more alkaline environment and milder acidic pH values which then will typically range from about 3.5 to about 6.0.

Via the creation of these milder acidic conditions, the stomach acids then present can still efficiently digest ingested food solids and beverages, but the milder acidic environment generated by the concentrated vinegar will concomitantly create far less cellular destruction and inflammation within the esophagus lining, and markedly reduce both the severity and the longevity of heartburn and/or symptoms of GERD.

Second, acetic acid is known to be a relatively weak organic acid. Chemically, this means that a part of the acetic acid content exits as an undissociated salt, a form that helps buffer and maintain stomach acids at a very desirable pH value of about 3.5-6.0. By creating and maintaining such a milder acid environment, the stomach can still efficiently digest food and liquids, but can also markedly diminish heartburn symptoms and the severity of an existing reflux problem.

Third, the acetic acid in the undiluted concentrated vinegar will influence and affect the function of the lower esophageal sphincter (LES), which is believed to be a pH sensitive muscular valve. Frequently when there is food in the stomach coupled with an insufficient quantity of hydrochloric acid for digestion, the LES valve can periodically open and allow the stomach contents to reflux upwards; and consequently, when the LES valve senses less hydrochloric acid, the LES tends to open and thereby initiate an acid reflux episode. Thus, orally ingesting a carefully controlled quantity of concentrated vinegar will supply the LES valve with a limited increase in acidity sufficient to induce the LES to function properly and remain closed.

### B. At Least One Undiluted Bioactive Honey

The medicament of the present invention requires that at least one undiluted bioactive honey having demonstrable non-peroxide antibacterial activity be used as an ingredient in the fluid blending. Accordingly, not less than five different requirements must be demonstrably present for the particular honey before it is deemed to be suitable for use as an ingredient. These requirements are:

(1) Regardless of type or source, the honey must be a natural or a mostly natural product;

(2) Regardless of type or source, the honey must exist and be used in undiluted form;

(3) Regardless of type or source, the honey must be demonstrably bioactive in-situ and possess substantial antibacterial activity;

(4) Regardless of type or source, the mechanism of action for the honey's in-situ antibacterial activity is non-peroxide based; and

(5) Regardless of type or source, the honey must retain demonstrable non-peroxide antibacterial activity after being combined with a concentrated vinegar.

### Proportional Ratio Range for the Ingredient

In general, not less than about 1.0% (w/v) and not more than about 44% (w/v) of the fluid blending is undiluted bioactive honey. This 1.0%-44% (w/v) proportional ratio range, however, is merely the broadest range deemed to be useful in preparing the medicament. A more desirable proportional ratio range is from about 3.0% to about 35% undiluted bioactive honey; and a highly preferred proportional ratio range is from about 8% to about 21% (w/v) undiluted bioactive honey.

Clearly therefore, in almost all formulations, the undiluted bioactive honey ingredient is neither the predominate nor the majority ingredient in the fluid blending of the medicament.

It will be noted and appreciated also that this specifically limited proportional ratio range is markedly unlike conventionally known honey preparations where the overwhelming predominate or majority ingredient is honey, rather than any other substance.

### The Three Conventionally Recognized Categories of Honey

In general, three different categories of honey are recognized to exist in nature. These are: (i) Regular honey; (ii) Ordinary Manuka honey; and (iii) Active Manuka honey. Each of these categories is described in detail below.

### Regular Honey

All types of regular honey, regardless of source, have some intrinsic antibacterial activity, due primarily to the presence of the enzyme glucose oxidase, which generates hydrogen peroxide in a “slow-release” manner. Because the antibacterial activity is directly dependent upon the quantity of hydrogen peroxide produced, and because the quantity of hydrogen peroxide actually formed by the glucose oxidase enzyme in any regular honey can be quite variable, the demonstrable antibacterial activity of any particular honey will vary greatly in potency. Thus, some regular honeys are no more antibacterial than table sugar (i.e., sucrose); while other types can be mildly diluted with water and still demonstrably halt the growth of bacteria. The recognized differences in the potency of antibacterial activity among the different types of regular honey are quite large and often substantial.

Regular honeys also collectively share some notable properties and distinguishing characteristics. These commonly shared attributes include all of the following:
All regular honeys have only very low levels of methylglyoxal, a highly potent non-peroxide antibacterial agent; and thus are actually dependent upon a very different biochemical activity basis and antibacterial mechanism of action—the enzymatic production of hydrogen peroxide in substantial quantities;
In all regular honeys, the glucose oxidase enzyme generates hydrogen peroxide in a “slow-release” manner, and it is the chemical action of the hydrogen peroxide, which provides the recognized antibacterial activity;
The glucose oxidase enzyme (which generates hydrogen peroxide in a “slow-release” manner) in regular honeys is destroyed by exposure to light, or by exposure to heat, or by reactive contact with protein-digesting enzymes found in wound fluids;
The glucose oxidase enzyme of regular honeys requires the presence of oxygen in order for the enzyme to generate hydrogen peroxide;
The generated hydrogen peroxide (and its antibacterial activity) of regular honeys is destroyed by the catalase enzyme which is normally present in human body tissues and blood serum, and thus whenever catalase enzyme is present the antibacterial activity of the honey becomes markedly diminished or is entirely eradicated; and
The generated hydrogen peroxide (and its antibacterial activity) of regular honeys requires that such acids as are then present within the honey be neutralized by fluids, even though the act of neutralizing the acids by fluid concomitantly dilutes the honey.

### Ordinary Manuka Honeys

Ordinary Manuka honeys (and some of its Australian equivalents) are honeys that contain relatively small quantities of Manuka nectar, but have predominate sources of nectar other than manuka. Ordinary Manuka honeys are thus quite similar to Regular honeys, but have been found to include a very low level of additional non-peroxide antibacterial components.

It is now known that the chemical basis and mechanism of action for such non-peroxide antibacterial activity is due to the combined action of Methylglyoxal (“MGO”) and an as yet unidentified synergistic component(s). The investigations reported to date show that there are two important aspects to this combination: (a) The synergistic components function to double the antibacterial effects of Methylglyoxal in-situ; and (b) In the absence of Methylglyoxal, the synergistic components also exert their own substantive antibacterial actions in-situ [see for example http://bio.waikato.ac.nz/honey/where.shtml].

However, only very small quantities of MGO are found in Ordinary Manuka honey. Thus, although some types of Ordinary Manuka honey have been reported to show some non-peroxide antibacterial activity, such bioactivity is at an extremely minimal level.

Instead, the primary mechanism responsible for the antibacterial activity of Ordinary Manuka honey is also the presence of the enzyme glucose oxidase, and its generation of hydrogen peroxide in a “slow-release” manner. Ordinary Manuka honey, regardless of type or source, therefore shares the same deficiencies as Regular honeys. For completeness of understanding, these deficiencies are once again recited below.
Ordinary Manuka honeys have very small quantities of Methylglyoxal (MGO) and thus are dependent upon the enzymatic production of hydrogen peroxide in substantial quantities as the chemical basis and mechanism of action for its antibacterial properties;
In all Ordinary Manuka honeys, the glucose oxidase enzyme generates hydrogen peroxide in a “slow-release” manner, and it is the chemical action of the released hydrogen peroxide, which alone provides the recognized antibacterial activity;
The glucose oxidase enzyme (which generates hydrogen peroxide in a “slow-release” manner) in Ordinary Manuka honeys is destroyed by exposure to light, and/or by exposure to heat, and/or by reactive contact with protein-digesting enzymes found in wound fluids;
The glucose oxidase enzyme of Ordinary Manuka honeys requires oxygen as a reactant in order for the enzyme to generate hydrogen peroxide as a product;
The generated hydrogen peroxide (and its antibacterial activity) of Ordinary Manuka honeys is destroyed by the catalase enzyme (which is normally present in human body tissues and blood serum), and thus whenever catalase enzyme is present the antibacterial activity of the Ordinary Manuka honey becomes markedly reduced or is lost entirely;
The generated hydrogen peroxide (and its antibacterial activity) of Ordinary Manuka honeys requires that such free acids as are then normally present within the honey be neutralized by fluids, even though the act of neutralizing the free acids by fluid concomitantly dilutes the effectiveness of the Ordinary Manuka honey.

### Active Manuka Honeys

The title “Active Manuka Honey” was adopted to identify and distinguish Manuka honey produced from Leptospermum species as being a very different and unique type of honey which has a substantially elevated antibacterial activity that is not dependent upon the production or action of hydrogen peroxide; in order to separate and distinguish this kind of honey from what was being sold as ordinary Manuka honey that did not have such an enhanced antibacterial activity. The distinguishing term “Active Manuka Honey” has served this purpose and been in use commercially and in many printed publications since 1998 [see for example http://manukahoneyus.com].

The unique non-peroxide antibacterial activity of Active Manuka honey is caused by the combined action of methylglyoxal (MGO) and an unidentified synergistic component(s). Unlike the very low levels of MGO found in Regular honeys and Ordinary Manuka honey, the unusually high quantities of MGO normally present in Active Manuka honey (produced from Leptospermum species) is unique—as is the presence of the as yet unidentified synergistic agents, which serve to more than double the antibacterial activity of MGO in-situ.

Research studies of different varieties of manuka honey collected from many sites over a large area of New Zealand have shown that the concentration of the MGO (and the unique non-peroxide type of antibacterial activity) in Active Manuka honey depends to some degree on the variety of manuka from which the honey is collected, but rests mainly on the markedly higher proportional ratio of manuka nectar existing in these particular kinds of honey. Thus, Regular honeys and Ordinary Manuka honeys which do not have substantial concentrations of MGO (and thus do not demonstrate the unique non-peroxide type of antibacterial activity) are believed to be produced predominantly from nectar sources other than manuka nectar [see for example http://honey.bio.waikato.ac.nz].

Equally important, the unique non-peroxide antibacterial activity provided by Active Manuka honeys is not affected by the catalase enzyme commonly present in body tissue and serum. It will be recalled that the catalase enzyme will break down hydrogen peroxide to a large degree, the major antibacterial factor found in the other categories of honey. Thus, if a honey without the unique non-peroxide antibacterial activity of Active Manuka honey were used to treat an infection, the potency of the other honey's antibacterial activity would be greatly reduced because of the breakdown action of the catalase enzyme.

Active Manuka honeys also have a number of other unique properties and characteristics. Among the more notable of these are the following:
Active Manuka honeys have approximately twice the antibacterial effect of Regular honeys.
Active Manuka honeys remain stable and biochemically active when exposed to heat, or light, or the absence of oxygen.
The Methylglyoxal of Active Manuka honeys diffuses deeper into human skin tissues than does the hydrogen peroxide released from other kinds of honey.
Active Manuka honeys have the ability to stimulate cytokine production and release from monocytes, long recognized as the beginning of normal cell multiplication and tissue repair.
Active Manuka honeys have a direct anti-inflammatory effect in-situ.
Active Manuka honeys are about twice as effective as other honey against Escherichia coli and Enterococci, common causes of infection in wounds, and are much more effective than other honeys against Helicobacter pylori (a common cause of peptic ulcers).
The physical properties of Active Manuka honeys play a part in its effectiveness as a wound dressing. Primarily because of its viscosity, Active Manuka honey provides a protective barrier, which prevents cross-infection.
Because of its osmolarity and its ability to draw fluid out from living tissues, Active Manuka honeys create a moist healing environment which provides optimum healing as new tissue growth not slowed by drying; thus, fibroblasts in-situ are able to pull the wound closed, and new epithelial cells grow level with the patient's skin surfaces such that no pitts or scarring results. Osmotically induced outflow also creates “drainage”, a flushing away of any harmful substances from bacterial contaminants from the wound.
The sugar content of Active Manuka honeys also aids in the rapid removal of malodour from wounds—as bacteria use glucose in preference to amino acids, and thus produce lactic acid instead of bad-smelling amines and mercaptans. Staphylococcus aureus is one of the pathogenic species most sensitive to the antibacterial activity of Active Manuka honeys; and there are many published reports of a complete inhibition of Staphylococcus aureus by Active Manuka honeys, even when diluted into much lower concentrations, thereby demonstrating the importance of these antibacterial factors.

### Types and Sources of Natural Undiluted Bioactive Honeys

Several different types and sources of undiluted bioactive honeys having substantive non-peroxide antibacterial activity are thus available for use as an ingredient in the fluid blending of the medicament. These different types and sources include the following:

1. Any Active Manuka honey, which is produced by a Leptospermum species and has substantive non-peroxide antibacterial activity.

2. Any UMF® (or “Unique Manuka Factor”) rated honey which has at least a rating of UMF® 1+, and preferably is rated in the range from about UMF® 10+ to about UMF® 35+. The UMR® rating system provides a comparative measurement or assessment of non-peroxide antibacterial activity without specifying the chemical agent or mechanism of action responsible for the antibacterial activity; and is comparison based on the known antibacterial properties of the common antiseptic, phenol. Thus for example, a type of honey that has a UMF® value of 10 is said to have the same degree of antibacterial activity as a 10% (w/v) solution of phenol.

3. Any MGO™ Certified honey having at least a rating of MGO™ 30+, and which preferably has a higher certified rating up to MGO™ 550+ or greater.

The MGO™ certification identifies what is the minimal content (in mg/kg units) of Methylglyoxal then present in the individual honey, which demonstrably shows a marked non-peroxide antibacterial activity. Thus, a honey certified as MGO™ 100+Manuka honey contains at least one hundred milligrams per kilogram of Methylglyoxal; and a Manuka honey certified as MGO™ 550+ contains at least 550 milligrams per kilogram of Methglyoxal.

4. Any Regular honey, which has been demonstrably fortified with not less than about 30 milligrams per kilogram of Methylglyoxal (MGO).

It is noted also that the addition of sufficient quantities of Methylglyoxal (MGO) to the Regular honey may be made as a chemically pure Methglyoxal compound; or be made as a mixed fraction of different chemical compounds which collective demonstrate substantive non-peroxide antibacterial activity (as described by U.S. Patent Application Publication No. 2008/0292715, the text of which is expressly incorporated by reference herein). Such MGO fortified Regular honeys are deemed to be the functional equivalent of a UMF rated Manuka honey.

5. Any Ordinary Manuka honey which has been demonstrably fortified with not less than about 100 milligrams per kilogram of Methylglyoxal.

Here also, the addition of Methylglyoxal (MGO) to the Ordinary Manuka honey may be made as a chemically pure Methglyoxal compound; or be made as a mixed fraction extract of different chemical compounds having demonstrable non-peroxide antibacterial activity (as described by U.S. Patent Application Publication No. 2008/0292715, the text of which is expressly incorporated by reference herein). Such MGO fortified Ordinary Manuka honeys are deemed to be the functional equivalent of a UMF rated Manuka honey.

### C. At Least One Flavoring Agent

In general, any type, kind or source of natural fruit flavoring is preferred and is very desirable for use in the fluid blending of the medicament. In this respect, it is of no importance what the particular flavoring is; or what is the chemical composition of the chosen flavoring; or whether or not the particular flavoring agent is a pure substance or a impure mixture of multiple compounds; or whether or not the chosen flavoring agent includes one or more other entities or extraneous substances in addition to the natural flavor extract.

Similarly, it is of no relevance whether the natural flavoring agent is or is not produced in-house by the manufacturer of the medicament; or is obtained as a commercial product made and sold by others. Neither is it material whether or not the flavoring agent is an extract, or a concentrate, or a distillation; nor whether or not it is in solid or liquid form; nor whether or not it is freshly made, frozen, freeze-dried, evaporated, or condensed. All of these mattes are deemed to be matters of commercial cost and/or personal preference. Furthermore, in many use instances, two or more different natural flavoring agents can and should be used in combination for best results.

It will be recognized also that, if and when it appears prudent to use them—one or more artificial flavoring agents can be used in place of a natural flavoring agent. The use of artificial flavoring agents, however, is deemed to be a last resort and the least desirable choice.

The proportional ratio range of all the flavoring agents—regardless of whether only a single agent is employed or multiple agents in combination are used—can vary in proportional ratio range from as little as 0.5% (w/v) to as much as 10% (w/v) of the fluid blending. This 0.5%-10% (w/v) proportional ratio range, however, is merely the broadest range deemed to be useful in preparing the medicament. A more desirable proportional ratio range is from about 2.0% to about 8.5% flavoring agent; and a highly preferred proportional ratio range is from about 3.3% to about 5.75% (w/v) flavoring agent.

### Representative And Illustrative Examples

Merely to demonstrate the acceptable range and to illustrate the substantial variety of suitable natural flavoring agents, the following representative examples are provided.
(i) Fruit flavorings such as peach extracts, pineapple syrups, apple pie with “crust” extracts, blueberry extracts, raspberry syrups, lime extracts, black cherry syrups, citric fruit extracts, and the like.
(ii) Maple syrups and molasses.
(iii) Mint flavorings such as spearmint and peppermint concentrates.
(iv) Cream, butter, and cheese flavorings.
(v) Coffee, tea, and chocolate concentrates.
(vi) Vegetable, plant and nut extracts, flavorings or oils.
(vii) Candy flavorings.
(viii) Liquor extracts and flavorings.
(ix) Spices and spice extracts, flavorings, or oils.
(x) Vanilla extracts, honey extracts, vinegar extracts, and rose oil extracts.
(xi) Astringency controlling flavors.

### D. At Least One Additional Sweetener

It is highly preferred that natural additional sweeteners, rather than primarily artificial or chemically synthesized additional sweeteners, be used whenever possible in the fluid blending; and a wide range and variety of additional sweeteners are conventionally known and commercially sold today.

Furthermore, in any embodiment of the medicament, the proportional ratio range of additional sweetener may vary from as little as about 1.0% (w/v) to as much as about 30% (w/v) of the fluid blending. This 1.0%-30% (w/v) proportional ratio range, however, is merely the broadest range deemed to be useful in preparing the medicament. A more desirable proportional ratio range is from about 2% to about 20% additional sweetener; and a highly preferred proportional ratio range is from about 5% to about 12% (w/v) additional sweetener.

Accordingly, merely illustrating the better-known natural sweeteners commonly available today are those representative examples presented below.

### Erythritol

This sweetener has a transparent white brilliant appearance and is a free-flowing crystalline powder. It has a very clean, sweet taste similar to sucrose with no significant after-taste. The dry form exhibits a strong cooling effect; and has a similar look and taste to sugar. Erythritol will brown like sugar. In sweetness, erythritol is only about 70% as sweet as white sugar (sucrose); and has fewer calories than white sugar (less than 0.2 calories per gram, only 5% as much as sucrose). Erythritol is a sugar alcohol that is not a source of “impact carbohydrates” that raise blood sugar, and is deemed to be suitable for low-carb (carbohydrate-restricted) diets.

### Sweeteners Derived from the Stevia Plant

The genus Stevia consists of 240 species of plants native to South America, Central America, and Mexico—with several species of plants found as far north as Arizona, New Mexico, and Texas. Human use of the sweetener species S. rebaudiana originated in South America; and it is recognized that the leaves of the stevia plant typically have 30-45 times the sweetness of sucrose (ordinary table sugar).

Conventionally known processes for isolating Rebaudioside A from the stevia plant will result in a product that delivers the desired sweetness without a bitter aftertaste. However, the known processes for isolating Rebaudioside a have a relatively high economic cost, which has unfortunately diminished commercial interest in its production.

Nevertheless in 2007, the Coca-Cola Company announced plans to obtain approval for Rebiana (isolated Rebaudioside A) for use as a food additive within the United States, and presented plans to market Rebiana-sweetened products in 12 countries that allow the stevia plant's use as a food additive. Subsequently in May 2008, the Coca-Cola Company and Cargill announced the availability of “Truvia”, a consumer brand of stevia sweetener containing erythritol and Rebiana, which the FDA approved as a food additive on Dec. 2^{nd}, 2008.

It will be noted and appreciated also that “Rebiana” is a trade name for a zero-calorie sweetener containing mainly the steviol glycoside rebaudioside A (Reb-A), which is extracted from the stevia plant. “Truvia” is the consumer brand name (or trademark) for a sweetener made of erythritol and “Rebiana”, a product marketed by Cargill. Another version of a similar product is “PureVia” (sold by PepsiCola Inc.); as is “Merisant”, another brand name of isolated Rebaudioside A.

### Rice Syrup

This product is the traditional Asian sweetener. Brown rice syrup is made from rice starch converted into maltose, a complex sugar. Rice syrup is the mildest-flavored of the liquid sweeteners and contains trace amounts of B vitamins and minerals. It is commonly used in cooking and baking, to sweeten hot or cold beverages and cereals, or as a spread for fresh breads.

### Sorghum Syrup

This sweetener originates from sorghum cane juice, which is then boiled to a syrup. Sorghum cane tends to need few pesticides, owing to its natural insect resistance.

### Sucanat

Sucanat is organically grown, freshly squeezed sugar cane juice, evaporated by a special Swiss process. In its natural state, it is highly nutritious because the molasses is not removed. The flavor is quite extraordinary sweet.

### Fructose

Fructose is the form of sugar that is primarily found in fruit. Fructose sugar is similar to common white sugar, but is significantly sweeter; and is degraded more slowly because it is broken down by an enzyme in the bowel, rather than by insulin. For this reason, fructose is deemed to be a safer sugar for diabetics, hyperglycemics and hypoglycemics.

### Agave Nectar

This product is a natural liquid sweetener, which is commonly available in three different grades: Light, medium and amber. Light agave is sweet but neutral, making it desirable for recipes where stronger flavor may interfere with taste. The flavor of agave becomes more intense and earthy with the darker grades. Agave is extracted from the agave plant, and is low on the glycemic index. It is about 1.5 times sweeter than refined sugar.

### Barley Malt

This product is a dark, sticky and boldly flavored syrup. It is composed primarily of maltose, a complex sugar that enters the bloodstream slowly. This sweetener also provides trace amounts of eight vitamins and several minerals.

### Date Sugar

Although not actually a sugar in the conventional sense, date sugar is a sweetener ground from dehydrated dates. Date sugar can be exchanged measure for measure for ordinary white sugar (sucrose).

### Corn Syrup

The process for making high fructose corn syrup (HFCS) out of corn was developed in the 1970s; and the use of high fructose corn syrup has grown rapidly, from less than three million short tons in 1980 to almost 8 million short tons in 1995. Today Americans consume more HFCS than white sucrose sugar.

High-fructose corn syrup (HFCS) is commercially produced by processing cornstarch to yield glucose; and then processing the glucose to produce a high percentage of fructose. In short, via somewhat complicated process, white cornstarch is turned into crystal clear syrup having a final concentration of about 55 percent fructose—what the industry calls high fructose corn syrup.

Lastly, it will be recognized also that, if and when it appears prudent to use them—one or more artificial additional sweeteners can be used in place of any of these natural additional sweeteners. The use of artificial flavoring agents, however, is deemed to be a last resort and the least desirable choice.

### II. The Variety of Formulations Using the Requisite Ingredients

The medicament comprising the present invention (regardless of whether it is an all-natural, natural, organic, or primarily artificial fluid blending) can be formulated in many diverse modes and different proportional ratios of ingredients.

A. In this regard, the complete medicament can and should be formulated to provide a lineage of different ingredient concentrations or strengths, by which the medicament is available to the human consumer as minimal, average and maximum strength fluid blendings. Thus, as the strength of the formulation increases, there is a corresponding increase in the therapeutic value of the medicament for the human consumer. Consequently, it is expected and intended that formulations having lesser percentages of concentrated vinegar (the “lower strength” formulations) will be employed as prophylactic and preventative medicaments, while those formulation having greater percentages of concentrated vinegar (the “higher strength” formulations) will be more effectively used as therapeutic and remedial medicaments.

It is again emphasized that not less than about 44% (w/v) and not more than about 97% (w/v) of the medicament (without regard to whether the fluid blending is all-natural, natural, organic, or primarily artificial) is concentrated vinegar—where the concentrated vinegar then employed has an acetic acid content ranging from not less than about 2.5% to not generally more than about 6.0% (w/v). Clearly therefore, concentrated vinegar always is the overwhelmingly predominate, if not actual majority ingredient in each formulation of the medicament; and the “strength” of the medicament is a reflection of and corresponds with the true percentage ratio of concentrated vinegar in that formulation.

B. In each instance and embodiment, and regardless of particular formulation, the medicament of the present invention (whether an all-natural, a natural, an organic, or a primarily artificial fluid blending) will demonstrate the following characteristics.

(i) The formulated medicament will have a determinable pH value ranging from about 2.5 to 6.0,

(ii) The formulated medicament will demonstrate a marked non-peroxide antibacterial activity in-situ, and

(iii) The formulated medicament is sufficiently palatable to the human mouth such that the medicament can be swallowed by a human subject without regurgitation or substantial gagging.

To illustrate the intended range of useful proportional ratios in these formulations and different embodiments, Table 1 below identifies the minimal and maximum ranges for the essential ingredients forming the fluid blending of the medicament.

TABLE 1
Ingredient Ratio Ranges

| | Minimal | Maximum |
|---|---|---|
| Ingredient | Percentage (w/v) | Percentage (w/v) |
| Concentrated Vinegar | 44.0% | 97.0% |
| Undiluted Bioactive Honey | 1.0% | 44.0% |
| Flavoring Agent | 0.5% | 10.0% |
| Additional Sweetener | 1.0% | 30.0% |

In addition, in order to appreciate better how diverse the proportional ratio of ingredients can be, a series of representative specific formulations is provided by Table 2 below; and exemplary ranges of the individual ingredients is presented by Table 3 below. It is clearly understood, however, that these particular formulations neither restrict nor limit the envisioned embodiments, but instead serve merely to illustrate what is the true range of fluid blending formulations, which are deemed to be useful and effective in-situ.

TABLE 2
Illustrative Preferred Formulations By Proportional
Ratio Percentage (w/v)

| Formula | (A) | (B) | (C) | (D) | (E) | (F) | (G) | (H) |
|---|---|---|---|---|---|---|---|---|
| ACV* | 45 | 70.5 | 74.5 | 78.5 | 82.5 | 84.5 | 87.5 | 89.5 |
| AMH^{#} | 24 | 20 | 16 | 12 | 8 | 6 | 3 | 1 |
| fruit flavor^{£} | 6 | 3 | 3 | 3 | 3 | 3 | 3 | 3 |
| cream^{§} | 3 | 1 | 1 | 1 | 1 | 1 | 1 | 1 |
| acer^{¥} | 2 | 0.5 | 0.5 | 0.5 | 0.5 | 0.5 | 0.5 | 0.5 |
| organoz^{¥} | 20 | 5 | 5 | 5 | 5 | 5 | 5 | 5 |
| pH | 3.48 | 3.33 | 3.29 | 3.24 | 3.22 | 3.21 | 3.20 | 3.18 |
| Formula | (I) | (J) | (K) | (L) | (M) | (N) | (O) | (P) |
| ACV* | 69 | 73 | 77 | 81 | 85 | 87 | 90 | 96.5 |
| AMH^{#} | 24 | 20 | 16 | 12 | 8 | 6 | 3 | 1 |
| fruit flavor^{£} | 0.5 | 0.5 | 0.5 | 0.5 | 0.5 | 0.5 | 0.5 | 0.5 |
| cream^{§} | 1 | 1 | 1 | 1 | 1 | 1 | 1 | 0.5 |
| acer^{¥} | 0.5 | 0.5 | 0.5 | 0.5 | 0.5 | 0.5 | 0.5 | 0.5 |
| organoz^{¥} | 5 | 5 | 5 | 5 | 5 | 5 | 5 | 1 |
| pH | 3.37 | 3.3 | 3.28 | 3.23 | 3.21 | 3.20 | 3.19 | 3.17 |

TABLE 3
Exemplary Formulations Of Individual Ingredients

| Formulation | ACV* | AMH^{#} | fruit flv^{£} | cream^{§} | acer^{¥} | organoz^{∈} |
|---|---|---|---|---|---|---|
| Minimalist | 41.3% | 1.4% | 0.50% | 0.50% | 0.29% | 20.20% |
| Median | 74.96% | 12.38% | 2.49% | 1.36% | 0.69% | 8.15% |
| Maximal | 96.1% | 28.5% | 5.50% | 2.80% | 1.80% | 1.00% |
| Useful | 60.96% | 8.74% | 1.55% | 0.78% | 0.23% | 7.74% |
| Optimal | 76.2% | 10.9% | 1.94% | 0.97% | 0.30% | 9.68% |
| Effective | 88.39% | 12.67% | 2.25% | 1.13% | 0.34% | 11.23% |

Where
ACV* is concentrated apple cider vinegar;
AMH^{#} is undiluted Active Manuka honey;
fruit flavor^{£} is any natural fruit flavoring agent;
cream^{§} is a natural cream-texture agent;
acer^{¥} is a natural astringency-away flavor which reduces the bite of the acetic acid (i.e., astringency); and
organoz^{∈} is a natural additional sweetener.
Indicated pH values are all measured at 20° C.

C. It is again emphasized here that the medicament of the present invention can be prepared as either an all-natural formulation; or as a natural product; or as a completely organic composition of matter; or as a primarily artificial and synthetic fluid blending.

Note that by definition:

(i) an all-natural medicament includes any medical substance, formulation, composition, or preparation that contains no artificial compounds or chemically synthesized ingredients; while

(ii) a natural medicament encompasses any medicinal substance, formulation, composition, or preparation comprised primarily of ingredients that exist in or are created by nature, but which includes one or more additional compounds, enhancements, fractions, or chemically synthesized materials in small proportional ratio quantities totaling less than about 25% of the matter; and that

(iii) an organic medicament is any substance, formulation, composition, or preparation that exists in the state or form created by nature and has been obtained without the use of either artificial methods or chemically synthesized compositions.

Accordingly, among these three format choices, the natural medicament is most preferred, while the all-natural medicament and the organic medicament are each equally desirable as a second choice.

In the alternative, should the seller or manufacturer wish it, a primarily artificial or chemically synthetic version of the formulated medicaments can also be produced at will without difficulty. It is noted, however, that the reason typically given for manufacturing a primarily artificial version of the medicament is the far lower cost of using synthetic ingredients. Nevertheless, despite these alleged cost savings, the preparation of an artificial medicament formed primarily of synthetic substances is deemed to be the least desirable manner for preparing any formulated medicament of the present invention.

### III. Optional, but Often Desirable, Additional Ingredients

A variety of entirely optional, but often desirable, additional ingredients may be combined with the essential ingredients to form the fluid blending of the medicament. The following examples merely illustrate the range and variety of representative optionally employed additional ingredients.

For the concentrated vinegar:

Water or oil soluble liquids having a pH range of 2.5 to 6.0;

Water or oil soluble liquid having an acetic acid content of 3.0% to 8.0%; and

Additional concentrated acetic acid.

For the undiluted bioactive honey:

Any kind or type of honey that has demonstrable antibacterial/antibiotic capabilities; and

Any soluble substance or admixture of substances that has demonstrable antibacterial/antibiotic capabilities.

For the flavoring agents:

Astringency controlling components, flavors, extracts, and oils;

Texture controlling components, flavors, extracts, and oils; and pH reducers.

For the additional sweeteners:

Raw sugar, table sugar, sugar, sugar alcohols, milk sugars;

Acesulfame; and

Synthetic sweeteners.

### IV. The Manipulative Steps Comprising the Method

The present invention provides preferred methods for naturally treating heartburn symptoms or an acid reflux episode in a living human subject. In the alternative, however, a method for treating heartburn symptoms or an acid reflux episode in a living human subject using an artificial medicament comprised primarily of synthetic substances is also available; but it is again emphasized here that the use of a primarily artificial medicament in these methods is deemed to be a least desirable choice.

Accordingly, each instance of using a preferred natural treatment method comprises four manipulative steps. Each of these steps is described in detail below.

Step 1: Obtaining a preferred all-natural, natural, or organic medicament comprising a fluid blending of at least one concentrated vinegar made by the fermentation of a fruit or fruit sugar, or a vegetable, or a grain; at least one undiluted bioactive honey which has substantial non-peroxide antibacterial activity and retains demonstrable non-peroxide antibacterial activity after being combined with said concentrated vinegar; at least one natural flavoring agent; and at least one additional natural sweetener, whereby the fluid blending of the natural medicament

(i) has a determinable pH value ranging from about 2.5 to 6.0,

(ii) demonstrates substantial non-peroxide antibacterial activity in-situ, and

(iii) is sufficiently palatable to the human mouth such that said medicament can be swallowed by a human subject without substantial gagging.

Note that the preferred all-natural, natural or organic medicament is intended to be a prepared-in-advance product which can be manufactured in bulk, dispensed into individual containers, and then stored indefinitely (but having a “best used by” shelf life of time) until needed for use. Then, if and when heartburn or symptoms of GERD appear, the human subject will have the prepared medicament on hand and ready to use as a medical treatment.

Step 2: Orally ingesting an effective quantity of the medicament on at least one treatment occasion.

Typically, the human subject will drink and swallow about two teaspoons (about 10 milliliters) of the preferred all-natural, natural or organic medicament on each treatment occasion, usually after each meal or at the occurrence of a heartburn or acid reflux event. Oral ingestion of the formulated medicament is made without substantial gagging, regurgitation, or other major difficulty or discomfort. Once swallowed, the preferred all-natural, natural or organic medicament will coat the cellular lining of the esophagus and then enter the stomach of the human subject.

Also, a single treatment occasion is considered to be the minimal number of instances when the medicament will be orally ingested. Realistically however, it is expected that the human subject will orally ingest two teaspoons (about 10 milliliters) of the medicament on two to three occasions daily as a preventative measure; and swallow two teaspoons (about 10 milliliters) of the medicament every three to four hours on a daily basis as a therapeutic treatment for severe cases of heartburn and symptoms of GERD.

Step 3: Allowing the orally ingested medicament to react with the stomach contents of the living human subject such that

(i) the ingested medicament causes and maintains a milder acidic pH value for the stomach of the human subject, and

(ii) the ingested medicament exerts non-peroxide antibacterial activity within the esophagus and stomach of the human subject.

It is commonly recognized that during instances of heartburn and GERD, the typical pH value of the stomach contents is about 2.0 to 3.0—a very harsh acid condition. The introduction of the ingested medicament will raise the pH value of the stomach then existing and create a more alkaline environment; and thereby generate much milder acidic pH values in the stomach which often will range from about pH 3.5 to about pH 5.0.

Owing to these much milder acidic conditions, the stomach acids will then still efficiently digest ingested food solids and beverages, but the milder acidic environment generated by the medicament will cause far less cellular damage and inflammation within the esophagus lining, and markedly reduce both the duration and severity of heartburn and/or symptoms of GERD. In addition, the lower esophageal sphincter at the top of the stomach will keep the contents of the stomach better contained such that the reliance on the affect of gravity is greatly diminished.

It is also well recognized that an acid reflux event can be the effect of a sudden rush of stomach contents into the esophagus and throat particularly during sleep. The affect of the ingested natural medicament on the stomach contents significantly reduces or, in most cases, eliminates this explosive action.

There is substantial documentation recommending acid reflux sufferers elevate the head of their bed as a method to get symptom relief. The natural medicament is significant in its benefit in such situations; in particular, within a day or two of treatment patients are able to sleep flat without experience of an acid reflux event.

Step 4: Determining that the severity of the heartburn or the acid reflux episode has become markedly reduced.

The afflicted human subject will receive the beneficial effects of the ingested medicament almost immediately. The individual should feel relief from heartburn quickly (typically within 1 minute or less); and the duration of effective amelioration is expected routinely to continue for three or four hours time thereafter, or until a food or liquid of the type that re-activate the symptoms is consumed, or until a human behavior or act that is known to re-activate the symptoms occurs.

As previously noted, saliva has a natural effect on minor heartburn; and there is experience, subsequent to taking the medicament, that the ‘saliva effect on heartburn’ returns to normal after ingesting the natural medicament of the present invention.

Equally important, the symptoms of GERD will become substantially diminished and subsequently neutralized over time as a direct consequence of the stomach reacting with the ingested medicament. Thus, the classic symptoms of an acid reflux episode (heartburn, chest pain especially while lying down, sour taste in the mouth, coughing, wheezing, hoarseness & sore throat, and regurgitation of food or liquid) will become less severe, be of shorter duration, and become less frequent in occurrence—after reactive contact with the natural medicament.

It will be recognized and appreciated that the issue for the afflicted patient is consumption of re-activating substances. Those patients that limit their intake of or are able to eliminate re-activating substances have a far better chance of full recovery from the symptoms.

Note also that those afflicted patient who continue to consume re-activating substances (even on a limited basis) may or may not recover fully from the symptoms, and may need to continue routine treatment with the natural medicament. Consequently, those patients failing to limit re-activating substances can expect ongoing symptoms subsequent to consumption of a re-activating substance. In such an event, the natural medicament will resolve the symptom(s) on each treatment occasion.

### V. The Different Modes and Manners of Using the Treatment Methodology

It will be recognized and appreciated that the present treatment methodology can be beneficially employed in different modes and alternative manner of use. Some illustrative variations as to such usage are presented below.

### A. Prophylactic And Therapeutic Treatment Regimens

The present methodology can be employed as a preventative measure prior to the onset of heartburn or GERD symptoms—i.e., as a prophylactic treatment method; or as a remedial measure—i.e., as a therapeutic treatment method after the occurrence of heartburn or an acid reflux episode.

### Prophylactic Treatment Regimens

As a preventative technique, it is very desirable that the afflicted human subject orally ingests the prepared-in-advance medicament on a fixed treatment schedule. In this manner, sufficient quantities of the medicament would be present at fairly regular intervals within the esophagus and stomach prior to the onset of any symptoms of either heartburn or GERD.

As merely one illustrative example of such a prophylactic treatment regimen, the individual would swallow two tablespoons of a formulated medicament about thirty or forty minutes before eating a meal. This quantity of an ingested medicament will cause and maintain a milder acidic pH value for the stomach of the human subject before eating a meal, and allow the ingested natural medicament to exert its unique non-peroxide antibacterial activity within the esophagus and the stomach of the human subject prior to the ingestion of any solid food.

In the alternative, the afflicted individual may choose to orally ingest an effective dose of the medicament on an unscheduled or whim basis. In such instances, the human subject will swallow the medicament whenever and wherever he decides it is appropriate to do so, for his own personal reasons. Despite the irregularity of this technique, the orally ingested medicament will function effectively and serve as a preventative measure to reduce the symptoms of heartburn and GERD whenever such an episode subsequently occurs thereafter.

### Therapeutic Treatment Regimens

As a therapeutic measure, the human subject will swallow an effective dose of the natural medicament as soon as possible after the outbreak of a heartburn-inducing event or an acid reflux episode.

As merely one illustrative example of such a remedial treatment regimen, the individual would swallow two tablespoons of a formulated medicament whenever the symptoms of heartburn or GERD occur. This quantity of ingested medicament will be effective to react immediately with the esophagus lining and the stomach contents; cause and maintain a milder acidic pH value for the stomach of the human subject; and allow the ingested medicament to exert its unique non-peroxide antibacterial activity within the esophagus and the stomach of the human subject. In this manner, the actions of the ingested medicament will control the symptoms as well as reduce the severity and duration of the heartburn and/or acid reflux episode.

### B. Quantitative Dosages & Frequency Of Oral Administration

Formulated fluid blendings of the medicament can be administered and orally ingested in any manner, which delivers them to the stomach of the human subject. The prepared medicament (regardless of whether it is an all-natural, natural, or organic, or primarily artificial fluid blending) can be introduced by any means or routing equipment that allows the natural medicament to react with the stomach contents such that a milder acidic pH value is achieved and maintained in the stomach of the human subject, and the ingested natural medicament exerts its non-peroxide antibacterial activity within the stomach of the human subject.

The dosage of the formulated medicament to be orally ingested by any living human patient will of course vary with and be dependent upon the age, overall health, and weight of the recipient; the kind of concurrent treatment, if any; the frequency of concurrent treatment; and the physician's current prognosis for the patient.

In general however, a quantity of medicament ranging from about 2 to about 3 milliliters per kilogram of body weight, in twice daily or three times daily administrations is expected to be effective to yield the desired preventative or therapeutic result. The true quantity of natural medicament to be ingested for effective results will vary directly with the severity of the heartburn or GERD, but should always be enough to insure that there is a sufficient concentration of natural medicament to cause and maintain a milder acidic pH value within the stomach [i.e., to balance the harsh pH value of about pH 2.0-2.5 and initiate a milder acidic stomach pH value from about 3.5 to about 5.0] for a measurable duration of time; as well as for the ingested natural medicament to exert its unique non-peroxide antibacterial activity in-situ within the esophagus and stomach of the human subject.

For best results, the overall duration of prophylactic or therapeutic treatment should be continued so long as a favorable clinical result is obtained. It is believed that this treatment regimen will exert antibacterial activity within the esophagus; will stimulate tissue repair in the esophagus; will cause a mild alkaline effect on the existing highly acid contents of the stomach; and markedly reduce the severity, duration, and frequency of heartburn and/or an acid reflux episode. However, it is as yet unclear whether or not this treatment method will eventually provide for complete absence of heartburn or GERD symptoms. For this reason especially, the treatment duration and dosage quantity of medicament should be carefully controlled and monitored.

### Possible Side Effects, Cautions & Contraindications

Initially, it is important to realize that the medicament of the present invention is a natural food product; and as such, generally will cause no more side effects than the consumption of any food fit for human consumption.

It is possible that some persons may experience signs of an allergic reaction—such as a rash, hives, itching, swelling of the mouth or throat, wheezing, or difficulty breathing—owing to the effect of the concentrated vinegar in the medicament. Generally however, such sensitized persons would have a similar result from consuming any vinegar-containing product.

Also, some persons may report incidence of constipation; while others might report the opposite effect, a loose stool or even diarrhea. It is believed, however, that such incidences as this have more to do with the type of food consumed by the person around the time of treatment, and has far less to do with the treatment using the natural medicament of the present invention.

Lastly, it is strongly recommended that diabetics consult with their physician prior to ingesting the medicament. This recommendation is based on the fact that the natural medicament contains methylglyoxal, a known digestive issue for diabetics.

### VI. The Beneficial Outcomes and Advantageous Results Provided by the Treatment Methodology

A. In order to appreciate properly what are the beneficial outcomes and advantageous results of the medicament and the alternative treatment methods, it is useful to consider what the human afflicted with heartburn and/or GERD is faced with today.

Currently available pharmaceuticals for treatment of heartburn or GERD all come with a recommended period of use. For example, prescription Omeprazole is recommended for use over a period of 8 weeks. The over-the-counter formulated version, “Prilosec”, is sold for 14 day and 28 day courses of treatment.

Also, even the simple and least expensive antacids, calcium and sodium bicarbonate, can have substantial side effects when over used. The alternative groups of antacids are aluminum hydroxide, magnesium hydroxide, or combinations of both; and these also can have substantial side effects if over used by the patient.

Furthermore, the major side effects of H2-receptor antagonists and proton pump inhibitors appear in many published reports and are significant in their effects.

Clearly then, for the patient with repetitive heartburn, acid reflux, GERD, or sour stomach issues—there comes the time when they should not, and often can no longer, use these conventional treatments on an ongoing basis. At that stage of disorder progression, even though such conventionally available drugs and treatments will generally stop the symptoms with regular daily use—nevertheless, when the treatment is discontinued, the symptoms re-occur.

In effect, the responsible patient is fundamentally trapped. As the symptoms continue, the patient's treatment choices are few: Either continue to use the conventional treatments beyond the recommended treatment periods and suffer the consequences of major side effects and drug complications; or discontinue the use of conventional drugs and suffer from recurring episodes of heartburn and/or GERD. Neither choice is a good one for the afflicted patient.

B. The medicament and the prophylactic and therapeutic modes of treatment provide a meaningful and highly significant new option for the afflicted patient. Two substantive points should always be remembered: First, the medicament is food fit for human consumption, and will be digested as food by the gastro-intestinal system of the human subject. Second, the medicament can be taken on a regular and continuous basis which is not time limited.

In addition to the foregoing, among the very desirable outcomes and consequences of using the treatment methods comprising the present invention are those major benefits and unexpected advantages identified below.

1. The ingested medicament exerts a broad antibacterial activity against many different kinds of bacterial present within the esophagus and the stomach;

2. The ingested medicament will stimulate tissue repair for injuries and wounds existing in the esophagus and stomach;

3. The ingested natural medicament provides nutrients—vitamins, amino acids and sugars—for existing cells and new cell proliferation in the esophagus and stomach;

4. The ingested medicament exerts a marked anti-inflammatory effect in the esophagus and stomach;

5. The ingested medicament exerts an alkaline effect within and causes a milder acidic environment (between pH 3.5-6.0) for the contents of the stomach;

6. The ingested medicament reduces the severity and the longevity of heartburn symptoms or an acid reflux episode;

7. The ingested medicament reduces the frequency of a heartburn or acid reflux episode;

8. The ingested medicament reduces the frequency of sour stomach; and

9. The ingested medicament will cause the LES valve to function properly and thus prevent new episodes of heartburn and acid reflux.

The present invention is not restricted in scope nor limited in form, except by the claims appended hereto.

What I claim is:

1. A medicament to be orally ingested by a living human subject for the treatment of a heartburn inducing event or an acid reflux episode, said medicament comprising a fluid blending of:
at least one concentrated vinegar;
at least one undiluted bioactive honey which has substantial non-peroxide antibacterial activity and retains its non-peroxide antibacterial activity after being combined with said concentrated vinegar;
at least one flavoring agent; and
at least one additional sweetener,
and whereby said medicament
(i) has a determinable pH value ranging from about 2.5 to 6.0,
(ii) demonstrates a marked non-peroxide antibacterial activity in-situ, and
(iii) is sufficiently palatable to the human mouth such that said medicament can be swallowed by a human subject without substantial gagging.

2. The medicament as recited in claim 1 wherein said medicament is selected from the group consisting of all-natural medicaments, natural medicaments, and organic medicaments.

3. The medicament as recited in claim 1 wherein said medicament is an artificial medicament formed primarily of synthetic substances.

4. The medicament as recited in claim 1 wherein said concentrated vinegar is made by the fermentation of one selected from the group consisting of a fruit sugar, a vegetable and a grain.

5. The medicament as recited in claim 1 wherein said concentrated vinegar comprises from about 44% to about 97% of the fluid blending.

6. The medicament as recited in claim 1 wherein said concentrated vinegar has a pH value ranging from about 2.5 and 4.0.

7. The medicament as recited in claim 1 wherein said undiluted bioactive honey has a pH value ranging from about 3.0 to 5.0.

8. The medicament as recited in claim 1 wherein said undiluted bioactive honey comprises from about 1% to about 44% of the fluid blending.

9. The medicament as recited in claim 1 wherein said non-peroxide antibacterial activity of said undiluted bioactive honey is effective against wound-infecting species of bacteria.

10. The medicament as recited in claim 1 wherein said undiluted bioactive honey is Active Manuka honey.

11. The medicament as recited in claim 1 wherein said undiluted bioactive honey is selected from the group consisting of a UMF® (or “Unique Manuka Factor”) rated honey which has at least a rating of UMF® 10+, a MGO™ Certified honey having at least a rating of MGO™ 100+, a Regular honey which has been demonstrably fortified with not less than about 100 milligrams per kilogram of Methylglyoxal (MGO), and an Ordinary Manuka honey which has been demonstrably fortified with not less than about 100 milligrams per kilogram of Methylglyoxal.

12. The medicament as recited in claim 1 wherein said flavoring agent comprises from about 0.5% to about 10% (w/v) of the fluid blending.

13. The medicament as recited in claim 1 wherein said flavoring agent is one selected from the group consisting of fruit flavorings, maple syrups, molasses flavors, mint flavorings, cream, butter and cheese flavorings, coffee, tea and chocolate concentrates, vegetable, plant and nut extracts, flavorings or oils, candy flavorings, liquor extracts and flavorings, spices and spice extracts, flavorings or oils, vanilla extracts, honey extracts, vinegar extracts, and rose oil extracts, and astringency controlling flavors.

14. The medicament as recited in claim 1 wherein said additional sweetener comprises from about 1.0% to about 30% (w/v) of the fluid blending.

15. The medicament as recited in claim 1 wherein said additional sweetener is one selected from the group consisting of erythritol, stevia and stevia derivatives, rice syrups, sorghum syrups, sucanat, fructose, agave nectar, barley malts, date sugars, and corn syrups.

16. A natural medicament to be orally ingested by a living human subject for the treatment of a heartburn inducing event or an acid reflux episode, said natural medicament comprising a fluid blending of:
concentrated apple cider vinegar;
an undiluted active manuka honey which has substantial non-peroxide antibacterial activity and retains demonstrable non-peroxide antibacterial activity after being combined with said concentrated apple cider vinegar;
at least one natural flavoring agent; and
at least one natural additional sweetener,
and whereby said medicament
(i) has a determinable pH value ranging from about 2.5 to 6.0,
(ii) demonstrates non-peroxide antibacterial activity in-situ, and
(iii) is sufficiently palatable to the human mouth such that said natural medicament can be swallowed by a human subject without substantial gagging.

17. An artificial medicament to be orally ingested by a living human subject for the treatment of a heartburn inducing event or an acid reflux episode, said artificial medicament being a fluid blending of primarily synthetic substances and comprising:
at least one concentrated vinegar;
at least one undiluted bioactive honey which has substantial non-peroxide antibacterial activity and retains its non-peroxide antibacterial activity after being combined with said concentrated vinegar;
at least one flavoring agent; and
at least one additional sweetener,
and whereby said artificial medicament
(i) has a determinable pH value ranging from about 2.5 to 6.0,
(ii) demonstrates a marked non-peroxide antibacterial activity in-situ, and
(iii) is sufficiently palatable to the human mouth such that said medicament can be swallowed by a human subject without substantial gagging.

18. A method for naturally treating a heartburn inducing event in a living human subject, said natural treatment method comprising the steps of:
obtaining a medicament comprising a fluid blending of
at least one concentrated vinegar made by the fermentation of one selected from the group consisting of a fruit sugar, a vegetable and a grain;
at least one undiluted bioactive honey which has substantial non-peroxide antibacterial activity and retains its non-peroxide antibacterial activity after being combined with said concentrated vinegar;
at least one natural flavoring agent; and
at least one natural additional sweetener,
and whereby said medicament
(i) has a determinable pH value ranging from about 2.5 to 6.0,
(ii) demonstrates a marked non-peroxide antibacterial activity in-situ, and
(iii) is sufficiently palatable to the human mouth such that said medicament can be swallowed by a human subject without substantial gagging;
orally ingesting an effective quantity of said natural medicament on at least one treatment occasion;
allowing said orally ingested medicament to react with the stomach contents of the living human subject such that
(a) said ingested medicament causes and maintains a milder acidic pH value for the stomach of the human subject, and
(b) said ingested medicament exerts non-peroxide antibacterial activity within the stomach of the human subject; and
determining that the severity of the heartburn inducing event has become markedly reduced.

19. A method for naturally treating an acid reflux episode in a living human subject, said treatment method comprising the steps of:
obtaining a medicament comprising a fluid blending of
at least one concentrated vinegar made by the fermentation of at least one selected from the group consisting of a fruit sugar, a vegetable and a grain;
at least one undiluted bioactive honey which has substantial non-peroxide antibacterial activity and retains demonstrable non-peroxide antibacterial activity after being combined with said concentrated vinegar;
at least one natural flavoring agent; and
at least one natural additional sweetener,
and whereby said medicament
(i) has a determinable pH value ranging from about 2.5 to 6.0,
(ii) demonstrates non-peroxide antibacterial activity in-situ, and
(iii) is sufficiently palatable to the human mouth such that said natural medicament can be swallowed by a human subject without substantial gagging;
orally ingesting an effective quantity of said medicament on at least one treatment occasion;
allowing said orally ingested medicament to react with the stomach contents of the living human subject such that
(a) said ingested medicament causes and maintains a milder acidic pH value for the stomach of the human subject,
(β) said ingested medicament exerts non-peroxide antibacterial activity within the stomach of the human subject; and
determining that the severity of the acid reflux episode has become markedly reduced.

20. The method as recited in claim 18 or 19 wherein said medicament is one selected from the group consisting of all-natural medicaments, natural medicaments, and organic medicaments.

21. The method as recited in claim 18 or 19 wherein said treatment is used as a prophylactic treatment.

22. The method as recited in claim 18 or 19 wherein said treatment is used as a therapeutic treatment.

23. The method as recited in claim 18 or 19 wherein said medicament is orally ingested on multiple treatment occasions.

24. A method for treating heartburn or an acid reflux episode in a living human subject, said treatment method comprising the steps of:
obtaining an artificial medicament formed as a fluid blending of primarily synthetic substances, wherein said artificial medicament comprises
at least one concentrated vinegar;
at least one undiluted bioactive honey which has substantial non-peroxide antibacterial activity and retains demonstrable non-peroxide antibacterial activity after being combined with said concentrated vinegar;
at least one flavoring agent; and
at least one additional sweetener,
and whereby said artificial medicament
(i) has a determinable pH value ranging from about 2.5 to 6.0,
(ii) demonstrates non-peroxide antibacterial activity in-situ, and
(iii) is sufficiently palatable to the human mouth such that said medicament can be swallowed by a human subject without substantial gagging;
orally ingesting an effective quantity of said medicament on at least one treatment occasion;
allowing said orally ingested medicament to react with the stomach contents of the living human subject such that
(α) said ingested medicament causes and maintains a milder acidic pH value for the stomach of the human subject,
(β) said ingested medicament exerts non-peroxide antibacterial activity within the stomach of the human subject; and
determining that the severity of the heartburn or acid reflux episode has become markedly reduced.
